(19) United States
(12) Patent Application Publication
(10) Pub. No.: US 2007/0048758 A1
(43) Pub. Date: Mar. 1, 2007

(54) IMPROVED PRIMER-BASED AMPLIFICATION METHODS

(75) Inventors: Sergey Lokhov, Kirkland, WA (US); Alan G. Mills, Marysville, WA (US); Eugene Lukhtanov, Bothell, WA (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: Epoch Biosciences, Inc., Bothell, WA (US)

(21) Appl. No.: 11/423,399

(22) Filed: Jun. 9, 2006

Related U.S. Application Data
(60) Provisional application No. 60/688,873, filed on Jun. 9, 2005.

(51) Int. Cl. C12Q 1/68; C12P 19/34

(52) U.S. Cl. 435/6; 435/91.2

(57) ABSTRACT

The present invention provides methods for amplifying a target nucleic acid with greater efficiency and accuracy by using one or more flap primers.

### CROSS-REFERENCE TO RELATED APPLICATIONS

This application claims the benefit of U.S. Provisional Patent Application No. 60/688,873, filed Jun. 9, 2005, the disclosure of which is incorporated by reference in its entirety for all purposes.

### FIELD OF THE INVENTION

The present invention relates to an improved amplification and detection system for nucleic acid sequence targets, including small nucleic acid targets (i.e., micro RNA (miRNA), small interfering RNA (siRNA)) and other small non-coding RNA's). The amplification assays comprise at least one primer with a 5′-non-complementary- and a 3′-complementary sequence portions, the later portion complementary to the target, and optionally detection probes. The system is used in methods of amplification and detection of target nucleic acids with increased efficiency and accuracy.

### BACKGROUND OF THE INVENTION

Available methods for the amplification and detection of target nucleic acid sequences include the use of the PCR amplification method (U.S. Pat. Nos. 4,683,195 and 4,683,195). Significant improvements of amplification and detection are the TaqMan (U.S. Pat. No. 5,487,972), Molecular Beacons (WO 95/13399), Invader Assay utilizing a flap probe (WO 98/42873) and MGB Eclipse amplification methods (WO 03/062445).

Misuhasi (J. Clin. Lab. Anal. 10: 277-284 and 285-93 (1996)) in a two part Technical Report, discussed the basic requirement for designing optimal PCR primers. Duplex Scorpion Primers (Solinas et al., Nucl. Acids Res., 29: e96 (2001), self-reporting PNA/DNA primers (Fiandaca et al., Genome Res., 11: 609-613 (2001) and wavelength-shifting primers (U.S. Pat. No. 6,037,130) have been reported.

Inclusion of universal bases (US Application 2003/0170711; Loakes D., Nucl. Acids Res. 29: 2437-47 (2001)), locked nucleic acids (Latorra et al., Biotechniques 34: 1150-1155(203)), substituted- and unsubstituted pyrazolo [3,4-d]pyrimidines and substituted pyrimidines (Belousov et al., Human Genomics, 1: 209-217 (2004)), promiscuous bases (U.S. application Ser. No. 10/954,955), 3-nitropyrrole and 5-nitroindole (Loakes et al., Nucl. Acids Res., 23: 2361-2366 (1995)) in amplification primers, have been reported.

Flap- or adapter- or overhang-primers have been reported by Becton, Dickenson and Company (U.S. Pat. Nos. 6,743,582, 6,316,200 and US patent publication 2003/016593) where the non-complementary flap- or adapter- or overhang sequence is hybridized to a detection probe. Potter in US application 2003/0207302 disclosed a universal probe system using a first primer with an overhang sequence and a second primer with an attachment means. Cloning and direct sequencing utilizing primer-adapter mediated PCR was reported by Espelund and Jacobsen (Biotechniques, 13: 74-81 (1992)). Generally, however, such flap primers have been used for detection and not amplification purposes.

Chen reported on the real-time PCR detection of miRNAs using stem-loop primers and a specific TagMan probe (Real-time profiling of miRNAs from single cells, EM Technologies Conference and Exhibition. Emerging Technologies of Drug Discovery, San Francisco. Apr. 5-6, 2005). The mirVana miRNA Detection Kit (mirVana™ miRNA Detection, Catalog #1552, Ambion, Austin, Tex., USA) for miRNA and siRNA is based on a simple solution hybridization using one or more radiolabeled RNA probes. Unhybridized RNA and excess probe are then removed by a rapid ribonuclease digestion step and analyzed on a denaturing polyacrylamide gel. Methods for quantifying the amount of a target nucleic acid of less than about 30 nucleotides length using two ligation domains that are complementary to the target nucleic acid has been reported (US application 2004/02591218). Dahlberg et al. disclosed the detection of small nucleic acids with the Invader assay (US application 2005/0074788). The Rolling Circle amplification of microRNA samples was reported in WO 2005/010159.

### BRIEF SUMMARY OF THE INVENTION

The present invention relates to compositions and methods for amplification, detection and characterization of nucleic acid targets including small nucleic acid targets (i.e., miRNA, siRNA, and other small non-coding RNAs). More particularly, the present invention relates to improved methods for the amplification, detection and quantitation of nucleic acid targets.

In a first aspect, the invention provides methods for amplification of a target nucleic acid in a sample, comprising:
a) contacting a sample suspected of containing the target nucleic acid with an amplification reaction mixture comprising at least one flap primer having the formula:
   5′-X—Y-3′ (I)
wherein X represents the 5′ sequence portion of the flap primer that is non-complementary to the target nucleic acid, Y represents the 3′ sequence portion of the flap primer that is complementary to the target nucleic acid, wherein X is from 3-40 nucleotides in length;
b) incubating the reaction mixture under amplification conditions; and
c) optionally detecting the amplified target nucleic acid.
In some embodiments, the methods further comprise:
(c) contacting the amplified target nucleic acid of step (b) with reaction mixture comprising:
(i) a primer comprising a sequence complementary to the target nucleic acid of step (b); and
(ii) a primer comprising a sequence complementary to the target nucleic acid of step (b) and a minor groove binder; and
(d) incubating the reaction mixture of step (c) under amplification conditions, thereby generating a second amplified target nucleic acid; and
(e) detecting the amplified target nucleic acid of step (d).

A method for amplification of a target nucleic acid in a sample, the method comprising:
(a) contacting the sample suspected of containing the target nucleic acid with an amplification reaction mixture comprising:
at least one flap primer comprising an annealed helper oligonucleotide and having the formula:
   5′-X—Y-3′
   3′-X′-5′ (II)
wherein X represents the 5′ sequence portion of the flap primer that is non complementary to the target nucleic acid, X′ represents the helper oligonucleotide sequence that is complementary to X and comprises at least one modified nucleoside base, and Y represents the 3′ sequence portion of the flap primer that is complementary to the target nucleic acid, wherein X is from 3-40 nucleotides in length;
(b) incubating the reaction mixture under amplification conditions, thereby generating an amplified target nucleic acid; and
(c) optionally detecting the amplified target nucleic acid.
In some embodiments the methods further comprise:
(c) contacting the amplified target nucleic acid of step (b) with reaction mixture comprising:
(i) a primer comprising a sequence complementary to the target nucleic acid of step (b); and
(ii) a primer comprising a sequence complementary to the target nucleic acid of step (b)and a minor groove binder; and
(d) incubating the reaction mixture of step (c) under amplification conditions, thereby generating a second amplified target nucleic acid; and
(e) optionally detecting the amplified target nucleic acid of step (d).

Another aspect of the invention provides a method for amplification of a target nucleic acid in a sample, the method comprising:
(a) contacting a sample suspected of containing the target nucleic acid with an amplification reaction mixture comprising:
a detection primer comprising a sequence complementary to the target nucleic acid, a minor groove binder, and fluorophore, wherein the fluorophore is quenched by the MGB and insertion of the MGB into a minor groove unquenches the fluorophore;
(b) incubating the reaction mixture under amplification conditions, thereby generating an amplified target nucleic acid; and
(c) optionally detecting the amplified target nucleic acid.

In some embodiments, the target nucleic acid is less than 30 nucleotide bases. In some embodiments, the target nucleic acid is DNA or RNA (e.g., mRNA, tRNA, rRNA, miRNA, or siRNA).

Generally, the methods produce an amplified target nucleic acid that produces a detectable signal that is at least about 1.25-fold to about 3 fold greater in comparison to the detectable signal from an amplified target nucleic acid amplified from an amplification reaction mixture that does not comprise at least one flap primer. Generally, the methods produce an amount of amplified target nucleic acid that is at least about 1.25-fold to about 3 fold greater in comparison to the amount of amplified target nucleic acid amplified from an amplification reaction mixture that does not comprise at least one flap primer. Further, the methods are particularly suited to continuous monitoring of a detectable signal.

These and other embodiments of the invention are further described by the detailed description that follows.

### DEFINITIONS AND ABBREVIATIONS

The following abbreviations are used in the present specification:

| | |
|---|---|
| MGB | minor groove binder |
| FL | fluorescent label or fluorophor |
| Q | quencher |
| CPG | controlled pore glass (as an example of a solid support) |
| ODN | oligonucleotide moieties or molecules |

Unless otherwise stated, the following terms used in the specification and claims have the meanings given below:

The terms “flap primer” or “overhang primer” refer to a primer comprising a 5′ sequence segment non-complementary to a target nucleic acid sequence and a 3′ sequence segment complementary to the target nucleic acid sequence. The flap primers of the invention are suitable for primer extension or amplification of the target nucleic acid sequence.

The term “overhang sequence” refers to a non-complementary adapter-, flap or overhang-sequence in a primer.

The term “helper oligonucleotide” refers to an oligonucleotide sequence complementary to at least a portion of the overhang sequence of a flap primer. A helper oligonucleotide binds to the overhang sequence and increases the specificity of an amplification reaction. In some embodiments, the helper oligonucleotide is complementary to the entire overhang sequence of the flap primer. In some embodiments, the helper oligonucleotide comprises at least one modified base (e.g., a super A or super T). In some embodiments, the helper oligonucleotide further comprises an MGB.

The terms “MGB-primer” and minor groove binder-primer” refer to an oligonucleotide comprising a sequence complementary to a target sequence of interest and having an attached minor groove binder. In some embodiments, the minor groove binder is covalently attached to the oliognucleotidetide.

The term “detection primer” refers to an oligonucleotide comprising a sequence complementary to a target sequence of interest and having both an attached minor groove binder (“MGB”) and an attached fluorophore. The MGB and fluorophore are both attached to the same end of the detection primer. When the detection primer is in solution, i.e., when the the MGB is not bound to the minor groove of a double stranded nucleic acid, the MGB quenches the signal from the fluorophore. When the MGB is bound to the minor groove of a double stranded nucleic acid, the fluorophore becomes unquenched and the signal from the fluorophore can be detected. In some embodiments, the minor groove binder and the fluorophore are both covalently attached to the oligonucleotide.

The term “target nucleic acid” refers to any nucleic acid sequence to be detected using the methods described herein. Suitable target nucleic acids include, e.g., DNA, mRNA, tRNA and rRNA, miRNA and siRNA. In some embodiments, the target nucleic acids are less than 50, 45, 40, 36, 30, 25, 20, 15, or 10 nucleotides in length.

As used herein, the term “miRNA” refers to micro RNA. As used herein, the term “miRNA target sequence” refers to a miRNA that is to be detected (e.g., in the presence of other nucleic acids). In some embodiments, a miRNA target sequence is a variant of a miRNA. Micro RNAs are reviewed, for example, in Ambros, Nature (2004) 431:350-5; Tang, Trends Biochem Sci (2005) 30:106-114; and Bengert and Dandekar, Brief Bioinform (2005) 6:72-85.

The term “siRNAs” refers to short interfering RNAs. In some embodiments, siRNAs comprise a duplex, or double-stranded region, where each strand of the double-stranded region is about 18 to 25 nucleotides long; the double-stranded region can be as short as 16, and as long as 29, base pairs long, where the length is determined by the antisense strand. Short interfering RNA is reviewed, for example, in Jones, et al., Curr Opin Pharmacol (2004) 4:522-7; and in Tang, supra.

A “sample” or “biological sample” include sections of tissues such as biopsy (e.g., from tissue suspected of being malignant) and autopsy samples, and frozen sections taken for histologic purposes. Such samples include blood, sputum, tissue, cultured cells, e.g., primary cultures, explants, and transformed cells, stool, urine, etc. A biological sample is typically obtained from a eukaryotic organism, most preferably a mammal such as a primate e.g., chimpanzee or human; cow; dog; cat; a rodent, e.g., guinea pig, rat, mouse; rabbit; or a bird; reptile; or fish.

An “amplification reaction” refers to any chemical reaction, including an enzymatic reaction, which results in increased copies of a template nucleic acid sequence or results in transcription of a template nucleic acid. Amplification reactions include reverse transcription and polymerase chain reaction (PCR), including Real Time PCR (see U.S. Pat. Nos. 4,683,195 and 4,683,202; PCR Protocols: A Guide to Methods and Applications (Innis et al., eds, 1990)). Exemplary “amplification reactions conditions” or amplification conditions” typically comprise either two or three step cycles. Two step cycles have a denaturation step followed by a hybridization/elongation step. Three step cycles comprise a denaturation step followed by a hybridization step followed by a separate elongation step. A temperature of about 36° C. is typical for low stringency amplification, although annealing temperatures may vary between about 32° C. and 48° C. depending on primer length. For high stringency amplification, a temperature of about 62° C. is typical, although high stringency annealing temperatures can range from about 50° C. to about 65° C., depending on the primer length and specificity. Typical cycle conditions for both high and low stringency amplifications include a denaturation phase of 90° C.-95° C. for 30 sec-2 min., an annealing phase lasting 10 sec.-2 min., and an extension phase of about 76° C. for 10 sec-2 min.

A “target nucleic acid” refers to a nucleic acid of interest that is in a sample. “Target nucleic acid” also reders to products of reverse transcription reactions and products of primer extension assays, either of which can be further amplified using the methods described herein.

In certain formulae, the group [A-B]n is used to refer to an oligonucleotide, modified oligonucleotide or peptide-nucleic acid having ‘n’ bases (B) and being linked along a backbone of ‘n’ sugars, modified sugars or amino acids (A).

The term “fluorescent generation probe” refers either a) to an oligonucleotide having an attached minor groove binder, fluorophore and quencher or b) DNA binding reagent.

The terms “fluorescent label” or “fluorophore” refers to compounds with a fluorescent emission maximum between about 400 and 900 nm. These compounds include, with their emission maxima in nm in brackets, Cy2™ (506), GFP (Red Shifted) (507), YO-PRO™-1 (509), YOYO™-1 (509), Calcein (517), FITC (518), FluorX™ (519), Alexa™ (520), Rhodamine 110 (520), 5-FAM (522), Oregon Green™ 500 (522), Oregon Green™ 488 (524), RiboGreen™ (525), Rhodamine Green™ (527), Rhodamine 123 (529), Magnesium Green™ (531), Calcium Green™ (533), TO-PRO™-1 (533), TOTO®-1 (533), JOE (548), BODIPY® 530/550 (550), Dil (565), BODIPY® TMR (568), BODIPY® 558/568 (568), BODIPY® 564/570 (570), Cy3™ (570), Alexa™ 546 (570), TRITC (572), Magnesium Orange™ (575), Phycoerythrin R&B (575), Rhodamine Phalloidin (575), Calcium Orange™ (576), Pyronin Y (580), Rhodamine B (580), TAMRA (582), Rhodamine Red™ (590), Cy3.5™ (596), ROX (608), Calcium Crimson™ (615), Alexa™ 594 (615), Texas Red® (615), Nile Red (628), YO-PRO™-3 (631), YOYO™-3 (631), R-phycocyanin (642), C-Phycocyanin (648), TO-PRO™-3 (660), TOTO®-3 (660), DiD DilC(5) (665), Cy5™ (670), Thiadicarbocyanine (671), Cy5.5 (694). Fluorophores further refers to fluorescent derivatives disclosed in WO 03/023357, WO 06/020947 and U.S. application Ser. No. 11/202,635.

The term “linker” refers to a moiety that is used to assemble various portions of the molecule or to covalently attach the molecule (or portions thereof) to a solid support. Typically a linker or linking group has functional groups that are used to interact with and form covalent bonds with functional groups in the ligands or components (e.g., fluorophores, oligonucleotides, minor groove binders, or quenchers) of the conjugates described and used herein. Examples of functional groups on the linking groups (prior to interaction with other components) include —NH₂, —NHNH₂, —ONH₂, —NHC═(O)NHNH₂, —OH, or —SH. The linking groups are also those portions of the molecule that connect other groups (e.g., phosphoramidite moieties and the like) to the conjugate. Additionally, a linker can include linear or acyclic portions, cyclic portions, aromatic rings or combinations thereof.

The term “solid support” refers to any support that is compatible with oligonucleotides synthesis, including, for example, glass, controlled pore glass, polymeric materials, polystyrene, beads, coated glass and the like.

The term “alkyl” refers to a linear, branched, or cyclic saturated monovalent hydrocarbon radical or a combination of cyclic and linear or branched saturated monovalent hydrocarbon radicals having the number of carbon atoms indicated in the prefix. For example, (C1-C8)alkyl is meant to include methyl, ethyl, n-propyl, 2-propyl, tert-butyl, pentyl, cyclopentyl, cyclopropylmethyl and the like. For each of the definitions herein (e.g., alkyl, alkenyl, alkoxy, aralkyloxy), when a prefix is not included to indicate the number of main chain carbon atoms in an alkyl portion, the radical or portion thereof will have eight or fewer main chain carbon atoms.

The term “alkylene” means a linear saturated divalent hydrocarbon radical or a branched saturated divalent hydrocarbon radical having the number of carbon atoms indicated in the prefix. For example, (C1-C6)alkylene is meant to include methylene, ethylene, propylene, 2-methylpropylene, pentylene, and the like.

The term “aryl” means a monovalent or bivalent (e.g., arylene) monocyclic or bicyclic aromatic hydrocarbon radical of 6 to 10 ring atoms which is unsubstituted or substituted independently with one to four substituents, preferably one, two, or three substituents selected from those groups provided below. The term “aryl” is also meant to include those groups described above wherein one or more heteroatoms or heteroatom functional groups have replaced a ring carbon, while retaining aromatic properties, e.g., pyridyl, quinolinyl, quinazolinyl, thienyl, and the like. More specifically the term aryl includes, but is not limited to, phenyl, 1-naphthyl, 2-naphthyl, thienyl and benzothiazolyl, and the substituted forms thereof.

Substituents for the aryl groups are varied and are selected from: -halogen, —OR′, —OC(O)R′, —NR′R″, —SR′, —R′, —CN, —NO₂, —CO₂R′, —CONR′R″, —C(O)R′, —OC(O)NR′R″, —NR″C(O)R′, —NR″C(O)₂R′, —NR′—C(O)NR″R′″, —NH—C(NH₂)═NH, —NR′C(NH₂)=NH, —NH—C(NH₂)═NR′, —S(O)R′, —S(O)₂R′, —S(O)₂NR′R″, —N₃, —CH(Ph)₂, perfluoro(C1-C4)alkoxy, and perfluoro(C1-C4)alkyl, in a number ranging from zero to the total number of open valences on the aromatic ring system; and where R′, R″ and R′″ are independently selected from hydrogen, (C1-C8)alkyl and heteroalkyl, unsubstituted aryl and heteroaryl, (unsubstituted aryl)-(C1-C4)alkyl, and (unsubstituted aryl)oxy-(C1-C4)alkyl.

Two of the substituents on adjacent atoms of the aryl or heteroaryl ring may optionally be replaced with a substituent of the formula -T-C(O)—(CH₂)_{q}—U—, wherein T and U are independently —NH—, —O—, —CH₂— or a single bond, and q is an integer of from 0 to 2. Alternatively, two of the substituents on adjacent atoms of the aryl or heteroaryl ring may optionally be replaced with a substituent of the formula -A-(CH₂)ᵣ—B—, wherein A and B are independently —CH₂—, —O—, —NH—, —S—, —S(O)—, —S(O)₂—, —S(O)₂NR′— or a single bond, and r is an integer of from 1 to 3. One of the single bonds of the new ring so formed may optionally be replaced with a double bond. Alternatively, two of the substituents on adjacent atoms of the aryl or heteroaryl ring may optionally be replaced with a substituent of the formula —(CH₂)ₛ—X—(CH₂)ₜ—, where s and t are independently integers of from 0 to 3, and X is —O—, —NR′—, —S—, —S(O)—, —S(O)₂—, or —S(O)₂NR′—. The substituent R′ in —NR′— and —S(O)₂NR′— is selected from hydrogen or unsubstituted (C1-C6)alkyl. Still further, one of the aryl rings (Ar1 and Ar2, below) can be further substituted with another substituted aryl group to extend the resonance ability of the aromatic system, directly or indirectly through groups such as —(CR′═CR′)ₙ— and —(C≡C)ₙ—, where n is 0 to 5, increasing the wavelength absorbance maximum.

The prefix “halo” and the term “halogen” when used to describe a substituent, refer to —F, —Cl, —Br and —I.

Certain compounds or oligonucleotides of the present invention may exist in a salt form. Such salts include base addition salts such as sodium, potassium, calcium, ammonium, organic amino, or magnesium salt, or a similar salt. When the compounds or modified oligonucleotides of the present invention contain relatively basic functionalities, acid addition salts can be obtained by contacting the neutral form of such compounds with a sufficient amount of the desired acid, either neat or in a suitable inert solvent. Examples of acceptable acid addition salts include those derived from inorganic acids like hydrochloric, hydrobromic, nitric, carbonic, monohydrogencarbonic, phosphoric, monohydrogenphosphoric, dihydrogenphosphoric, sulfuric, monohydrogensulfuric, hydriodic, or phosphorous acids and the like, as well as the salts derived from organic acids like acetic, propionic, isobutyric, maleic, malonic, lactic, benzoic, succinic, suberic, fumaric, mandelic, phthalic, benzenesulfonic, p-tolylsulfonic, citric, tartaric, methanesulfonic, and the like. Also included are salts of amino acids such as arginate and the like, and salts of organic acids like glucuronic or galactunoric acids and the like (see, for example, Berge, S. M., et al, “Pharmaceutical Salts”, Journal of Pharmaceutical Science, 1977, 66, 1-19). Certain specific compounds of the present invention contain both basic and acidic functionalities that allow the compounds to be converted into either base or acid addition salts.

The neutral forms of the compounds may be regenerated by contacting the salt with a base or acid and isolating the parent compound in the conventional manner. The parent form of the compound differs from the various salt forms in certain physical properties, such as solubility in polar solvents, but otherwise the salts are equivalent to the parent form of the compound for the purposes of the present invention.

Certain compounds of the present invention can exist in unsolvated forms as well as solvated forms, including hydrated forms. In general, the solvated forms are equivalent to unsolvated forms and are intended to be encompassed within the scope of the present invention. Certain compounds of the present invention may exist in multiple crystalline or amorphous forms. In general, all physical forms are equivalent for the uses contemplated by the present invention and are intended to be within the scope of the present invention.

Certain compounds of the present invention possess asymmetric carbon atoms (optical centers) or double bonds; the racemates, diastereomers, geometric isomers and individual isomers are all intended to be encompassed within the scope of the present invention. The methods for the determination of stereochemistry and the separation of isomers are well-known in the art (see discussion in Chapter 4 of “Advanced Organic Chemistry”, 4th edition J. March, John Wiley and Sons, New York, 1992).

The compounds of the present invention may also contain unnatural proportions of atomic isotopes at one or more of the atoms that constitute such compounds. For example, the compounds may be radiolabeled with radioactive isotopes, such as for example tritium (³H), iodine-125 (¹²⁵I) or carbon-14 (¹⁴C). All isotopic variations of the compounds of the present invention, whether radioactive or not (e.g, ²H), are intended to be encompassed within the scope of the present invention.

“Protecting group” or “protected form thereof” refers to a grouping of atoms that when attached to a reactive group in a molecule masks, reduces or prevents that reactivity. Examples of protecting groups can be found in T. W. Greene and P. G. Futs, Protective Groups in Organic Chemistry, (Wiley, 2nd ed. 1991) and Harrison and Harrison et al., Compendium of Synthetic Organic Methods, Vols. 1-8 (John Wiley and Sons. 1971-1996). Representative amino protecting groups include formyl, acetyl, trifluoroacetyl, benzyl, benzyloxycarbonyl (CBZ), tert-butoxycarbonyl (Boc), trimethyl silyl (TMS), 2-trimethylsilyl-ethanesulfonyl (SES), trityl and substituted trityl groups, allyloxycarbonyl, 9-fluorenylmethyloxycarbonyl (FMOC), nitro-veratryloxycarbonyl (NVOC) and the like. Representative hydroxy protecting groups include those where the hydroxy group is either acylated or alkylated such as benzyl and trityl ethers as well as alkyl ethers, tetrahydropyranyl ethers, trialkylsilyl ethers and allyl ethers. In general, the preferred protecting groups are those that can be removed under acidic conditions or basic conditions, or those groups that can be removed by the use of a particular light source (e.g., “light sensitive” protecting groups). Additionally, selection of an appropriate protecting group is made with due consideration to other functionality in the molecule so that either the incorporation or removal of the protecting group does not interfere or otherwise significantly affect the remainder of the molecule.

“Optional” or “optionally” in the above definitions means that the subsequently described event or circumstance may but need not occur, and that the description includes instances where the event or circumstance occurs and instances in which it does not. For example, “aryl optionally mono- or di-substituted with an alkyl group” means that the alkyl group may, but need not, be present, and the description includes situations where the aryl group is mono- or disubstituted with an alkyl group and situations where the aryl group is not substituted with the alkyl group.

The practice of the present invention will employ, unless otherwise indicated, conventional techniques in organic chemistry, biochemistry, oligonucleotide synthesis and modification, bioconjugate chemistry, nucleic acid hybridization, molecular biology, microbiology, genetics, recombinant DNA, and related fields as are within the skill of the art. These techniques are fully explained in the literature. See, for example, Sambrook and Russell, MOLECULAR CLONING: A LABORATORY MANUAL, Third Edition, Cold Spring Harbor Laboratory Press (2000); CURRENT PROTOCOLS IN MOLECULAR BIOLOGY, Ausubel, et al., eds., John Wiley & Sons (1987-2005, as cumulatively updated); Gait (ed.), OLIGONUCLEOTIDE SYNTHESIS: A PRACTICAL APPROACH, IRL Press (1984); Eckstein (ed.), OLIGONUCLEOTIDES AND ANALOGUES: A PRACTICAL APPROACH, IRL Press (1991); Herdewijn, OLIGONUCLEOTIDE SYNTHESIS: METHODS AND APPLICATIONS, Humana Press (2004); and Agrawal, PROTOCOLS FOR OLIGONUCLEOTIDES AND ANALOGS: SYNTHESIS AND PROPERTIES, Humana Press (1993).

The term “Eclipse™ probe” refers, in general, to a 5′-MGB-Q-ODN-FL probe. In contrast, a “TaqMan® MGB™ probe refers to a 3′-MGB-Q-ODN-FL probe. A “Pleiades probe” refers to a 5′-MGB-FL-ODN-Q probe. Eclipse™ and MGB™ are trademarks of Epoch Biosciences, Inc., Bothell, Wash.; and TaqMan® is a registered trademark of Applied Biosystems, Inc., Foster City, Calif.

### BRIEF DESCRIPTION OF THE DRAWINGS

FIG. 1. Schematic amplification of a nucleic target with overhang primers. X is a target non-complementary sequence portion and Y is a target complementary sequence.

FIG. 2a) The effect of flap primer sequence length on amplification signal, detected with a MGB Eclipse probe. b) The effect of flap primer sequence length on amplification signal, detected with a Pleiades probe. F designates a Flap and the number following indicates the length of the flap sequence. MGB is the DPI₃ ligand.

FIG. 3a) Comparison of the effect of the presence of flap sequence on amplification in a single or both primers detected with a MGB Eclipse probe. b) Comparison of the effect of the presence of flap sequence on amplification in a single or both primers detected with a Pleiades probe.

FIG. 4a) MGB Eclipse real-time PCR assay using normal and/or flap primers. b) Pleiades real-time PCR assay using normal and/or flap primers. The sequences of amplicon target, primers and probes are shown in Table 3.

FIG. 5a) MGB Eclipse real-time PCR assay using normal and/or flap primers. b) Pleiades real-time PCR assay using normal and/or flap primers. The sequences of amplicon target, primers and probes are shown in Table 4.

FIG. 6. MGB Eclipse RT-PCR amplification titration of human parainfluenza virus (1×10⁵ to 1×10⁰ copies of viral RNA) with different primer pairs. Real-time curves of primer pairs 13/17, 14_{F-12}/18_{F-12} and 16_{F-12}/19_{F-12} is shown in a), c) and e), respectively corresponding linear titration curves are shown in b), d) and f). The primer, probe and amplicon sequences are shown in Table 5. F designates a Flap and the number following indicates the length of the flap sequence.

FIG. 7a). Singleplex amplification of B2MG with normal and flap primers. b) Singleplex amplification of GI with normal and flap primers. c) Biplex amplification of B2MG and GI with no flap primers. d) Biplex amplification of B2MG and GI with flap primers.

FIG. 8a). Real-time amplification detection of hsa-miR-139 DNA target with SYBR Green detection. b) Melt curve analysis of amplified target.

FIG. 9 illustrates the reverse transcription and PCR amplification using three different primers, including one MGB-containing primer.

FIG. 10 a) real-time amplification of titration of synthetic hsa-miR-142-3P target with primer limiting primer #1 containing a DPI₂ moiety attached to the 5′end and b) limiting primer #2 containing a DPI₃ moiety attached to the 5′end.

FIG. 11 shows the real-time amplification of titration of synthetic hsa-miR-142-3P target with limiting primer #1 containing a DPI3 moiety attached to the 5′end and in the presence of helper x. FIG. 11A shows the results from real-time amplification of the synthetic hsa-miR-142-3P target using RT primer 4 and FIG. 11B shows the results from real-time amplification of the synthetic hsa-miR-142-3P target using RT primer 4a.

FIG. 12 illustrates the reverse transcriptase and PCR amplification of a short RNA target using three different primers, including one MGB-containing primer.

FIG. 13 shows the real-time amplification of hsa-miR-142-3P target. Reverse transcription performed with HL-60 total RNA (Strategene, La Jolla, Calif.). Four 5 fold dilutions of the cDNA underwent real-time PCR amplification. Limiting primer oligonucleotide 7, contains a 12 bp non-complementary sequence on the 5′end.

FIG. 14 shows the detection of a hsa-miR-142-3P target.

FIG. 15 shows real-time amplification of a synthetic hsa-miR-16-1 precursor molecule.

FIG. 16 shows real-time amplification of a) a synthetic mature hsa-miR-16 and b) an hsa-miR-16 mature sequence from a synthetic hsa-miR-16-1 precursor molecule. FIG. 16A shows the results from real-time amplification using primer #15 and FIG. 16B shows the results from real-time amplification using primer 15a.

FIG. 17 shows the melting curves of let-7a and let-7d amplicon templates probed with let-7a probe.

FIG. 18 illustrates how the MGB-Fl-Oligonucleotide primer functions as a detection moiety. As shown in FIG. 18, the the fluorescence of MGB-Fl-oligonucleotide primer is quenched by the MGB when unhybridized. However, once the MGB binds to the minor groove of an amplification product, the fluorophore is unquenched and fluoresces.

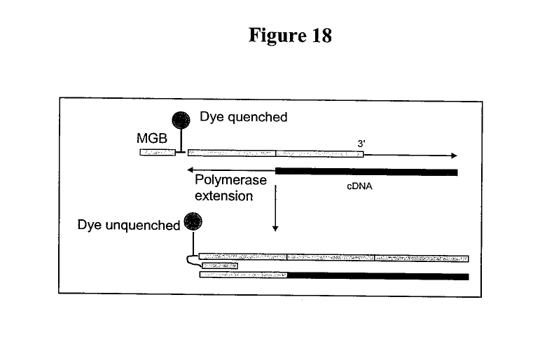

FIG. 19 illustrates reverse transcription and PCR amplification using the MGB-Fl-Oligonucleotide primer as a detection moiety

FIG. 20 shows a titration curve (10 fold dilutions) of hsa-miR-16 target with detection of 2.3×10⁷ to 23 copies.

FIG. 21a) Real-time plots for FAM-labeled probe for the miR-16 target bi-plexed with the Yakima Yellow-labeled probe for the 18S rRNA house keeping gene in HL-60 total RNA at 50 ng/reaction. b) Real-time plots for FAM-labeled probe for the miR-21 target bi-plexed with the Yakima Yellow-labeled probe for the 18S rRNA house keeping gene in HL-60 total RNA at 50 ng/reaction. Real-time data for the miR-16 and miR-21 targets was measured in the FAM-channel and that for 18S target in the YY-channel. “s” is singleplex and “b” is biplex.

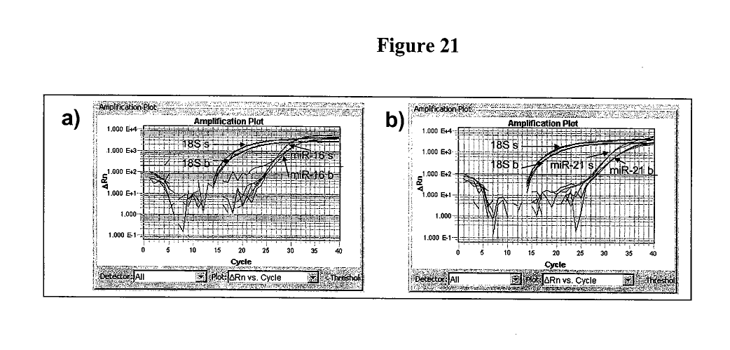

### DETAILED DESCRIPTION

I. General

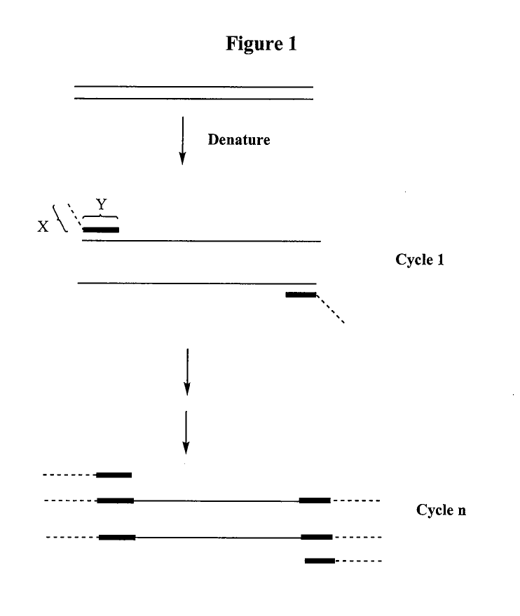

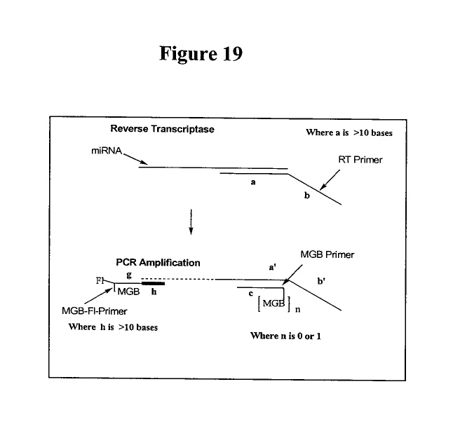

The invention is based on the surprising discover that primers containing an overhang sequence are particularly useful in the efficient and accurate amplification of nucleic acid targets. These primers are particularly useful in real-time amplification detection, for example, where the amplified target nucleic acid is detected simultaneously with amplification. Moreover, the overhang primers described herein display a significant improved signal compared to primers without an overhang sequence. Schematic representations of amplification with primers containing an overhang sequence are shown in FIGS. 1, 9, 12, and 19.

The primers of the present invention provide numerous advantages over existing primers in the amplification of nucleic acids and especially the amplification of short nucleic acid targets. The primers of the invention are particularly useful in allowing the efficient and accurate amplification and, optionally, detection of nucleic acid targets, for example using reverse transcription, primer extension, and PCR.

II. Amplification of Target Nucleic Acids

The invention provides methods for amplification of a target nucleic acid using flap primers. Amplification of a target nucleic acid includes generation of a amplified target nucleic acid following reverse transcription (RT) as well as amplification of the product of a reverse transcription reaction, e.g., by PCR. The RT and PCR reactions can be performed in two steps or as a single step.

In a first aspect, the invention provides methods for amplification of a target nucleic acid in a sample, comprising:
(a) contacting the sample susepected of containing the target nucleic acid with an amplification reaction mixture comprising at least one flap primer having the formula:
   5′-X—Y-3′ (I)
wherein X represents the 5′ sequence portion of the flap primer that is non-complementary to the target nucleic acid, Y represents the 3′ sequence portion of the flap primer that is complementary to the target nucleic acid, wherein X is from 3-40 nucleotides in length;
(b) incubating the reaction mixture under amplification conditions, thereby generating an amplified target nucleic acid; and
(c) optionally detecting the amplified target nucleic acid.

In some embodiments, the flap primer further comprise an annealed helper oligonucleotide and has the formula
5′-X—Y-3′
3′-X′-5′ (II)
wherein X represents the 5′ sequence portion of the flap primer that is non complementary to the target nucleic acid, X′ represents the helper oligonucleotide sequence that is complementary to at least a portion of X, and Y represents the 3′ sequence portion of the flap primer that is complementary to the target nucleic acid. In some embodiments, X′ comprises at least one modified base. (e.g., a super A, a super T, or super G). X′ may comprise few bases than X, more bases than X, or the same number of bases than X. In some embodiments, the helper oligonucleotide has a Tₘ of about 50° Cy.

In certain embodiments, the target nucleic acid can be DNA, mRNA, tRNA, rRNA, siRNA, or miRNA. In some embodiments, the target nucleic acid is less than 50, 45, 40, 35, 30, 35, 20, or 15 nucleotide bases. Nucleic acids of less than about 50 nucleotide bases are typically 10-50, 15-40, or 20-25 nucleotides in length, but can be about 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 nucleotides in length.

Generally, the methods produce an amount of amplified target nucleic acid that is at least 1.2-fold, 1.25-fold, 1.3-fold, 1.4-fold, 1.5-fold, 1.6-fold, 1.7-fold, 1.8-fold, 1.9-fold, 2.0-fold, 2.1-fold, 2.2-fold, 2.3-fold, 2.4-fold, 2.5-fold, 2.6-fold, 2.7-fold, 2.8-fold, 2.9-fold, or 3.0-fold greater in comparison to an amount of amplified target nucleic acid amplified from an amplification reaction mixture that does not comprise at least one flap primer.

Generally, the methods produce an amplified target nucleic acid that generates a detectable signal that is at least 1.2-fold, 1.25-fold, 1.3-fold, 1.4-fold, 1.5-fold, 1.6-fold, 1.7-fold, 1.8-fold, 1.9-fold, 2.0-fold, 2.1-fold, 2.2-fold, 2.3-fold, 2.4-fold, 2.5-fold, 2.6-fold, 2.7-fold, 2.8-fold, 2.9-fold, or 3.0-fold greater in comparison to an amplified target nucleic acid amplified from an amplification reaction mixture that does not comprise at least one flap primer. Further, the methods are particularly suited to continuous monitoring of a detectable signal (“real-time detection”). In certain embodiments, simultaneous amplification is detected using a fluorescence-generating detection probe, for example, a hybridization-based fluorescent probe or a DNA binding fluorescent compound.

In certain embodiments, the reaction mixture comprises two flap primers: a forward flap primer and a reverse flap primer. The forward flap primer and the reverse flap primer can be, but need not be, of equal lengths.

In certain embodiments, the 5′ sequence portion of the flap primer that is non-complementary to the target nucleic acid (X) is about 3-40, about 5-30, 7-20, 9-15 nucleotides in length, or about 10-14 or 11-13, or about 12 nucleotides in length. The 5′ sequence portion of the flap primer that is non-complementary to the target nucleic acid (X) can be 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40 nucleotides in length.

In certain embodiments, the 3′ sequence portion of the flap primer that is complementary to the target nucleic acid (Y) comprises a greater number of nucleotides than the 5′ sequence portion of the flap primer that is non-complementary to the target nucleic acid (X). For example, the 3′ sequence portion of the flap primer that is complementary to the target nucleic acid (Y) can comprise about 55%, 60%, 65%, 70%, 75%, 80%, 85% or 90% of the total length of a flap primer. In some embodiments, the 3′ sequence portion of the flap primer that is complementary to the target nucleic acid is 10, 11, 12, 13, 14, 15, 16, 17, 18, 19, 20, or more nucleotides in length.

In certain embodiments, the 5′ sequence portion of the flap primer that is non-complementary to the target nucleic acid (X) comprises about an equal number of nucleotides as the 3′ sequence portion of the flap primer that is complementary to the target nucleic acid (Y). For example, the X and Y portions each can be about 4-30, 6-25, 8-20, 10-15 nucleotides in length, usually about 10-14 or 11-13 nucleotides in length, and more usually about 12 nucleotides in length. The X and Y portions each can be 4, 5, 6, 7, 8, 9, 10, 11, 12, 13, 14, 15, 16, 17, 18, 19, 20, 21, 22, 23, 24, 25, 26, 27, 28, 29 or 30 nucleotides in length.

In certain embodiments, the 5′ sequence portion of the flap primer that is non-complementary to the target nucleic acid (X) comprises at least about 60%, 65%, 70%, 75%, 80%, 90%, 95% adenine or thymine nucleotide bases, or modified bases thereof. In certain embodiments, the 5′ sequence portion of the flap primer that is non-complementary to the target nucleic acid (X) comprises at least about 60%, 65%, 70%, 75%, 80%, 90%, 95% guanine or cytosine nucleotide bases, or modified bases thereof.

In certain embodiments, the methods further comprise amplifying the amplified target nucleic acid with a reaction mixture comprising a first primer comprising a covalently attached minor groove binder and a sequence complementary to the target nucleic acid and a second primer comprising a sequence complementary to the target nucleic acid and detecting the resulting amplification products. In some embodiments, the first primer comprises a sequence of about 5-20, 6-15, 8-12 or more than 10 bases that are complementary to the target nucleic acid. In some embodiments, the second primer comprises as equence of about 5-50, 7-40, 9-30, or 11-20 bases that are complementary to the target nucleic acid. In some embodiments, the second primer is a flap primer of Formula I or II, an MGB-primer (see, e.g., U.S. Pat. No. 6,312,894), or a detection primer. In some embodiments, the MGB is a DPI₂ or DPI₃ moiety. Other suitable MGB are set forth in U.S. Pat. No. 5,801,155 and U.S. Patent Publication No. 20050187383. MGB-primers have been disclosed in co-owned U.S. Pat. No. 6,312,894.

An amplified target nucleic acid can be detected using any of the methods of detection known in the art. For example, detection can be carried out after completion of an amplification reaction (e.g., using ethidium bromide in an agarose gel) or simultaneously during an amplification reaction (“real-time detection”). See, for example, PCR Primer: A Laboratory Manual, Dieffenbach, et al., eds., 2003, Cold Spring Harbor Laboratory Press; McPherson, et al., PCR Basics, 2000; and Rapid Cycle Real-time PCR Methods and Applications: Quantification, Wittwer, et al., eds., 2004, Springer-Verlag. In certain embodiments, the amplified target nucleic acid is detected using one or more fluorescence-generating detection probes. Fluorescence-generating detection probes include probes that are cleaved to release fluorescence (Taqman, nuclease IV), nucleic acid binding compounds (US application 2003/026133, U.S. Pat. Nos. 5,994,056, 6,171,785, Bengtsson et al. Nucl. Acids Res., 31: e45 (2003) and U.S. Pat. No. 6,569,627), hybridization-based probes (Eclipse, Molecular Beacons, Pleiades, etc.), and the like. In certain embodiments, the target nucleic acid is detected with one or more DNA binding fluorescent compounds (e.g., SYBR® Green 1 (Molecular Probes, Eugene, Oreg.), BOXTOX, BEBO (TATAA Biocenter, Gotenborg, Sweeden).

In one embodiment, a target nucleic acid of less than about 30 nucleotide bases in length is detected using a fluorescence-generating detection probe that hybridizes to the target nucleic acid and one or more nucleotide bases of at least one flap primer sequence (typically, the non-complementary portion, X). For example, the fluorescence-generating detection probe can hybridize to a target nucleic acid and to one or more nucleotide bases of the forward flap primer sequence, one or more nucleotide bases of the reverse flap primer sequence, or simultaneously to one or more nucleotide bases of both the forward and the reverse flap primer sequences. The fluorescence-generating detection probe can optionally hybridize to a target nucleic acid and to 1, 2, 3, 4, 5, 6, 7, 8, 9, or 10 nucleotide bases of at least one flap primer sequence, particularly the non-complementary portion, X, of a flap primer.

The primers of the invention can incorporate additional features which allow for the detection or immobilization of the primer but do not alter the basic property of the primer, that of acting as a point of initiation of nucleic acid synthesis. Biotinylated primers have been used to immobilize amplified target (Olsvik et al., Clin Microbiol Rev., 7: 43-54 (1994)). In some instances, the primers contain one or more non-natural bases or modified bases in either or both the complementary- and non-complementary sequence regions of the primer.

In certain embodiments, amplification is carried out using a polymerase. The polymerase can, but need not have 5′ nuclease activity. In certain embodiments, primer extension is carried out using a reverse transcriptase and amplification is carried out using a polymerase.

In one embodiment, the primer sequences overlaps, wherein the stability of the overlapping sequence duplex is less than that of the stability of the individual primer target duplexes.

A. Primers

In one aspect, the present invention provides “overhang primers”, “flap primers” or “adapter primers” which are most generally noted as 5′-X—Y-3′ primers. X represents the sequence portion of the primer non-complementary to the target and Y the target complementary sequence portion of the primer.

Accordingly, in one group of embodiments, the primer has the formula:
5′-X—Y-3′ (I)
in which X represents the 5′ sequence of the primer non-complementary to the target, Y the complementary 3′ sequence of the primer, X—Y represents the nucleic acid oligomer primer. In certain further embodiments, X is [A-B]ₘ, and Y is [A-B]ₙ, wherein A represents a sugar phosphate backbone, modified sugar phosphate backbone, locked nucleic acid backbone or a variant thereof used in nucleic acid preparation, B represents a nucleic acid base, a modified base of a base, the subscript m is an integer of from about 3-18 or 4-16, usually from about 8-15, 10-14 or 11-13, and more usually about 12. The subscript n is an integer from about 4 to 50, usually from 8-20, 10-18, or 12-16. In certain embodiments the values of the subscripts m and n are equal, for example, both m and n simultaneously can be an integer of about 8-15, 10-14 or 11-13, and more usually about 12.

In some embodiments, the flap primer further comprise an annealed helper oligonucleotide and has the formula
5′-X—Y-3′
3′-X′-5′ (II)
wherein X represents the 5′ sequence portion of the flap primer that is non complementary to the target nucleic acid, X′ represents the helper oligonucleotide sequence that is complementary to at least a portion of X, and Y represents the 3′ sequence portion of the flap primer that is complementary to the target nucleic acid.

In another embodiment, the invention provides detection primers comprising a sequence complementary to a target nucleic acid, an MGB, and a fluorophore. The MGB and the fluorophore are attached (e.g., covalently) to the same end of the primer. When the MGB and the fluorophore are at an appropriate proximity to each other, the MGB quenches the signal from the fluorophore, i.e., the MGB reduces the signal from the fluorophore by at least about 40%, 50%, 55%, 60%, 65%, 70%, 75%, 80%, 85%, 90%, 95%, or 100%. When the MGB is bound to the minor groove of a double stranded nucleic acid (e.g., an amplified target sequence), the fluorophore is unquenched and a signal is emitted. Detection of the signal detects the presence of the double stranded nucleic acid.

The primers of the present invention are generally prepared using solid phase methods known to those of skill in the art. In general, the starting materials are commercially available, or can be prepared in a straightforward manner from commercially available starting materials, using suitable functional group manipulations as described in, for example, March, et al., ADVANCED ORGANIC CHEMISTRY—Reactions, Mechanisms and Structures, 4th ed., John Wiley & Sons, New York, N.Y., (1992).

The primers of the invention can comprise any naturally occurring nucleotides, non-naturally occurring nucleotides, or modified nucleotides known in the art.

B. Oligonucleotides and Modified Oligonucleotides

The terms oligonucleotide, polynucleotide and nucleic acid are used interchangeably to refer to single- or double-stranded polymers of DNA or RNA (or both) including polymers containing modified or non-naturally-occurring nucleotides, or to any other type of polymer capable of stable base-pairing to DNA or RNA including, but not limited to, peptide nucleic acids which are disclosed by Nielsen et al. Science 254:1497-1500 (1991); bicyclo DNA oligomers (Bolli et al., Nucleic Acids Res. 24:4660-4667 (1996)) and related structures. The primers of the present invention can include the substitution of one or more naturally occurring nucleotide bases within the oligomer with one or more non-naturally occurring nucleotide bases or modified nucleotide bases so long as the primer can initiate amplification of a target nucleic acid sequence in the presence of a polymerase enzyme.

For example, the oligonucleotide primers may also comprise one or more modified bases, in addition to the naturally-occurring bases adenine, cytosine, guanine, thymine and uracil. Modified bases are considered to be those that differ from the naturally-occurring bases by addition or deletion of one or more functional groups, differences in the heterocyclic ring structure (i.e., substitution of carbon for a heteroatom, or vice versa), and/or attachment of one or more linker arm structures to the base. Preferred modified nucleotides are those based on a pyrimidine structure or a purine structure, for example, 7-deazapurines and their derivatives and pyrazolopyrimidines (described in, for example, WO 90/14353, U.S. Pat. No. 7,045,610 and U.S. Pat. No. 6,127,121).

Exemplified modified bases (B) for use in the present invention include the guanine analogue 6-amino-1H-pyrazolo[3,4-d]pyrimidin-4(5H)-one (ppG or PPG, also Super G) and the adenine analogue 4-amino-1H-pyrazolo[3,4-d]pyrimidine (ppA or PPA). The xanthene analogue 1H-pyrazolo[5,4-d]pyrimidin-4(5H)-6(7H)-dione (ppX) can also be used. These base analogues, when present in an oligonucleotide, strengthen hybridization and improve mismatch discrimination. All tautomeric forms of naturally-occurring bases, modified bases and base analogues may be included in the oligonucleotide conjugates of the invention. Other modified bases useful in the present invention include 6-amino-3-prop-1-ynyl-5-hydropyrazolo[3,4-d]pyrimidine-4-one, PPPG; 6-amino-3-(3-hydroxyprop-1-yny)1-5-hydropyrazolo[3,4-d]pyrimidine-4-one, HOPPPG; 6-amino-3-(3-aminoprop-1-ynyl)-5-hydropyrazolo[3,4-d]pyrimidine-4-one, NH₂PPPG; 4-amino-3-(prop-1-ynyl)pyrazolo[3,4-d]pyrimidine, PPPA; 4-amino-3-(3-hydroxyprop-1-ynyl)pyrazolo[3,4-d]pyrimidine, HOPPPA; 4-amino-3-(3-aminoprop-1-ynyl)pyrazolo[3,4-d]pyrimidine, NH₂PPPA; 3-prop-1-ynylpyrazolo[3,4-d]pyrimidine-4,6-diamino, (NH₂)₂PPPA; 2-(4,6-diaminopyrazolo[3,4-d]pyrimidin-3-yl)ethyn-1-ol, (NH₂)₂PPPAOH; 3-(2-aminoethynyl)pyrazolo[3,4-d]pyrimidine-4,6-diamine, (NH₂)₂PPPANH₂; 5-prop-1-ynyl-1,3-dihydropyrimidine-2,4-dione, PU; 5-(3-hydroxyprop-1-ynyl)-1,3-dihydropyrimidine-2,4-dione, HOPU; 6-amino-5-prop-1-ynyl-3-dihydropyrimidine-2-one, PC; 6-amino-5-(3-hydroxyprop-1-yny)-1,3-dihydropyrimidine-2-one, HOPC; and 6-amino-5-(3-aminoprop-1-yny)-1,3-dihydropyrimidine-2-one, NH₂PC; 5-[4-amino-3-(3-methoxyprop-1-ynyl)pyrazol[3,4-d]pyrimidinyl]-2-(hydroxymethyl)oxolan-3-ol, CH₃OPPPA; 6-amino-1-[4-hydroxy-5-(hydroxymethyl)oxolan-2-yl]-3-(3-methoxyprop-1-ynyl)-5-hydropyrazolo[3,4-d]pyrimidin-4-one, CH₃OPPPG; 4,(4,6-Diamino-1H-pyrazolo[3,4-d]pyrimidin-3-yl)-but-3-yn-1-ol, Super A; 6-Amino-3-(4-hydroxy-but-1-ynyl)-1,5-dihydro-pyrazolo[3,4-d]pyrimidin-4-one; 5-(4-hydroxy-but-1-ynyl)-1H-pyrimidine-2,4-dione, Super T; 3-iodo-1H-pyrazolo[3,4-d]pyrimidine-4,6-diamine ((NH₂)₂PPAI); 3-bromo-1H-pyrazolo[3,4-d]pyrimidine-4,6-diamine ((NH₂)₂PPABr); 3-chloro-1H-pyrazolo[3,4-d]pyrimidine-4,6-diamine ((NH2)2PPACl); 3-Iodo-1H-pyrazolo[3,4-d]pyrimidin-4-ylamine (PPAI); 3-Bromo-1H-pyrazolo[3,4-d]pyrimidin-4-ylamine (PPABr); and 3-chloro-1H-pyrazolo[3,4-d]pyrimidin-4-ylamine (PPACl).

In addition to the modified bases (B) noted above, the oligonucleotides of the invention can have a backbone of sugar or glycosidic moieties (A), preferably 2-deoxyribofuranosides wherein all internucleotide linkages are the naturally occurring phosphodiester linkages. In alternative embodiments however, the 2-deoxy-β-D-ribofuranose groups are replaced with other sugars, for example, β-D-ribofuranose. In addition, β-D-ribofuranose may be present wherein the 2-OH of the ribose moiety is alkylated with a C₁₋₆ alkyl group (2-(O—C₁₋₆ alkyl) ribose) or with a C₂₋₆ alkenyl group (2-(O—C₂₋₆ alkenyl) ribose), or is replaced by a fluoro group (2-fluororibose). Related oligomer-forming sugars useful in the present invention are those that are “locked”, i.e., contain a methylene bridge between C-4′ and an oxygen atom at C-2′. Other sugar moieties compatible with hybridization of the oligonucleotide can also be used, and are known to those of skill in the art, including, but not limited to, α-D-arabinofuranosides, α-2′-deoxyribofuranosides or 2′,3′-dideoxy-3′-aminoribofuranosides. Oligonucleotides containing α-D-arabinofuranosides can be prepared as described in U.S. Pat. No. 5,177,196. Oligonucleotides containing 2′,3′-dideoxy-3′-aminoribofuranosides are described in Chen et al. Nucleic Acids Res. 23:2661-2668 (1995). Synthetic procedures for locked nucleic acids (Singh et al, Chem. Comm., 455-456 (1998); Wengel J., Acc. Chem. Res., 32:301-310 (1998)) and oligonucleotides containing 2′-halogen-2′-deoxyribofuranosides (Palissa et al., Z. Chem., 27:216 (1987)) have also been described. The phosphate backbone of the modified oligonucleotides described herein can also be modified so that the oligonucleotides contain phosphorothioate linkages and/or methylphosphonates and/or phosphoroamidates (Chen et al., Nucl. Acids Res., 23:2662-2668 (1995)). Combinations of oligonucleotide linkages are also within the scope of the present invention. Still other backbone modifications are known to those of skill in the art. The incorportation of modified bases during oligonucleotide synthesis is known in the art and requires the use of cyanoethylphosphoramidites modified bases wherein the reactive groups are appropriately blocked with protecting groups (WO 90/14353, U.S. Pat. No. 7,045,610 and U.S. Pat. No. 6,127,121).

The ability to design probes and primers in a predictable manner using an algorithm, that can direct the use or incorporation of modified bases, minor groove binders, fluorphores and/or quenchers, based on their thermodynamic properties have been described in U.S. Patnt Publication No. 20030224359. Accordingly, the use of any combination of normal bases, unsubstituted pyrazolo[3,4-d]pyrimidine bases (e.g., PPG and PPA), 3-substituted pyrazolo[3,4-d]pyrimidines, modified purine, modified pyrimidine, 5-substituted pyrimidines, universal bases, sugar modification, backbone modification or a minor groove binder to balance the Tₘ (e.g., within about 5-8° C.) of a hybridized product with a modified nucleic acid is contemplated by the present invention.

C. Fluorescence-Generating Probes

The overhang-primer amplified nucleic acid targets of the invention can be conveniently detected by fluorescent generating probes. A variety of fluorescence based detection probes are known in the art.

1. Hybridization-Based Probes.

5′-Minor groove binder-quencher oligonucleotide-fluorophore-3′ probes (WO 03/062445 and Afonina et al., Biotechniques 32; 940-949 (2002)), 5′-Minor groove binder-fluorophore-oligonucleotide-quencher-3′ probes (U.S. application Ser. No. 10/976,365), molecular beacons (U.S. Pat. No. 5,118,801) and PNA molecular beacons (WO 99/22018) detect amplified nucleic acid target with hybridization-based fluorescence generation. The preferred MGB ligand is dihydropyrroloindole tripeptide (DPI₃). The minor groove binder technology has been disclosed in U.S. Pat. Nos. 5,801,155; 6,312,894; and 6,492,346 and that of the Eclipse Dark Quencher in U.S. Pat. Nos. 6,727,356 and 6,790,945. The construction of the different oligonucleotide conjugates requires the use of linker molecules. The preferred linker molecules has been disclosed in U.S. Pat. Nos. 5,419,966 and 5,512,667. The minor groove binder technology has been disclosed in U.S. Pat. Nos. 5,801,155; 6,312,894; and 6,492,346 and that of the Eclipse Dark Quencher in U.S. Pat. Nos. 6,727,356 and 6,790,945. The disclosures of each of the references recited in this paragraph are hereby incorporated herein by reference in their entirety for all purposes.

2. Cleavage Probes

3′-Minor groove binder-quencher-oligonucleotide-fluorophore-5′ probes (Kutyavin et al, Nucl. Acids Res., 28: 655-661 (2000)) are cleaved by the 5′-nuclease activity of polymerase to release fluorescence. WO 04/018626 discloses the detection of amplified target by the endonuclease IV cleavage of a quencher-oligonucleotide-fluorophore probe.

3. DNA Binding Agents

Homogeneous methods for amplified nucleic acid detection with nucleic acid binding reagents have been disclosed (U.S. Pat. Nos. 5,994,056, 6,171,785 and Bengtsson et al. Nucl. Acids Res., 31: e45 (2003)). SYBR® Green I, a DNA binding agent, has been used for monitoring amplification (U.S. Pat. No. 6,569,627).

III. Amplifications Using Flap Primers

The methods of the present invention comprise carrying out primer-based amplification using the flap primers of the invention. In general, the flap primers of the invention can be substituted for normal primers containing the same nucleotide sequence in primer-based amplification with no or little change in the amplification reaction conditions. In some embodiments, the complementary sequence portion of the flap primer can be shorter, than that of a corresponding non flap primer. One skill in the art will recognize that routine minor re-optimization of the reaction conditions may be beneficial in certain amplification reactions.

In a preferred embodiment, the flap primers of the present invention are used with PCR. However, the invention is not restricted to any particular amplification system, e.g. reverse transcriptase. The flap primers can be used in different amplification methods as described above and illustrated in the examples below.

The present invention is compatible with methods of reducing non-specific amplification. For example the primers of the invention can be reversibly blocked (U.S. Pat. No. 6,509,157) and used with a reversibly inactivated enzyme (U.S. Pat. Nos. 5,677,152 and 5,773,258), reversibly inactivated polymerase enzymes are commercially available.

The primers of the present invention are useful in other techniques in which hybridization of an oligonucleotide to another nucleic acid is involved. These include, but are not limited to, techniques in which hybridization of an oligonucleotide to a target nucleic acid is the endpoint; techniques in which hybridization of one or more oligonucleotides to a target nucleic acid precedes one or more polymerase-mediated elongation steps which use the oligonucleotide as a primer and the target nucleic acid as a template; techniques in which hybridization of an oligonucleotide to a target nucleic acid is used to block extension of another primer; and techniques in which two or more oligonucleotides are hybridized to a target nucleic acid and interactions between the multiple oligonucleotides are measured. Determination of conditions for hybridization of oligonucleotides and factors which influence the degree and specificity of hybridization, such as temperature, ionic strength and solvent composition, are well-known to those of skill in the art. See, for example, Sambrook et al., supra; Ausubel, et al., supra; M. A. Innis et al. (eds.) PCR Protocols, Academic Press, San Diego, 1990; B. D. Hames et al. (eds.) Nucleic Acid Hybridisation: A Practical Approach, IRL Press, Oxford, 1985; and van Ness et al. (1991) Nucleic Acids Res. 19:5143-5151. In still other methods, multiple probes can be used to detect alternate target site regions (e.g., to identify difficult sequences or to differentiate species and subspecies of the target).

Hybridization of primers or oligonucleotide probes to target sequences proceeds according to well-known and art-recognized base-pairing properties, such that adenine base-pairs with thymine or uracil, and guanine base-pairs with cytosine. The property of a nucleotide that allows it to base-pair with a second nucleotide is called complementarity. Thus, adenine is complementary to both thymine and uracil, and vice versa; similarly, guanine is complementary to cytosine and vice versa. An oligonucleotide which is complementary along its entire length with a target sequence is said to be perfectly complementary, perfectly matched, or fully complementary to the target sequence, and vice versa. An oligonucleotide and its target sequence can have related sequences, wherein the majority of bases in the two sequences are complementary, but one or more bases are noncomplementary, or mismatched. In such a case, the sequences can be said to be substantially complementary to one another. If the sequences of an oligonucleotide and a target sequence are such that they are complementary at all nucleotide positions except one, the oligonucleotide and the target sequence have a single nucleotide mismatch with respect to each other. For the purposes of the present invention, fully complementary and substantially complementary sequences are considered complementary.

For those primer and oligonucleotide probes which incorporate modified bases, it is understood that the modified bases will retain the base-pairing specificity of their naturally-occurring analogues. For example, PPPG analogues are complementary to cytosine, while PPPA analogues are complementary to thymine and uracil. The PPPG and PPPA analogues not only have a reduced tendency for so-called “wobble” pairing with non-complementary bases, compared to guanine and adenine, but the 3-substituted groups increase binding affinity in duplexes. Similarly, modified pyrimidines hybridize specifically to their naturally occurring counter partners.

Conditions for hybridization are well-known to those of skill in the art and can be varied within relatively wide limits. Hybridization stringency refers to the degree to which hybridization conditions disfavor the formation of hybrids containing mismatched nucleotides, thereby promoting the formation of perfectly matched hybrids or hybrids containing fewer mismatches; with higher stringency correlated with a lower tolerance for mismatched hybrids. Factors that affect the stringency of hybridization include, but are not limited to, temperature, pH, ionic strength, concentration of organic solvents such as formamide and dimethylsulfoxide and chaotropes.

The degree of hybridization of an oligonucleotide to a target sequence, also known as hybridization strength, is determined by methods that are well-known in the art. A preferred method is to determine the Tₘ of the hybrid duplex. This is accomplished by subjecting a duplex in solution to gradually increasing temperature and monitoring the denaturation of the duplex, for example, by absorbance of ultraviolet light, which increases with the unstacking of base pairs that accompanies denaturation. Tₘ is generally defined as the temperature midpoint of the transition in ultraviolet absorbance that accompanies denaturation. Alternatively, if Tₘs are known, a hybridization temperature (at fixed ionic strength, pH and solvent concentration) can be chosen that it is below the Tₘ of the desired duplex and above the Tₘ of an undesired duplex. In this case, determination of the degree of hybridization is accomplished simply by testing for the presence of hybridized probe.

In some embodiments, the primers of the invention and the degree of hybridization of the primers can also be determined by measuring the levels of the extension product of the primer. In this embodiment, either the primer can be labeled, or one or more of the precursors for polymerization (normally nucleoside triphosphates) can be labeled. Extension product can be detected, for example, by size (e.g., gel electrophoresis), affinity methods with hybridization probes as in real time PCR, or any other technique known to those of skill in the art.

Amplification of Micro RNA Sequences

In one embodiment, the known miRNAs of an organism or a subset of the miRNAs of the organisms are determined simultaneously with the methods of the invention. In a preferred embodiment, the miRNAs are analyzed in a microtiter plate format, for example, using 96-, 192-, 384-, 768-, or 1536-well plates. Micro RNA sequences for many organisms are listed in the miRNA registry and updated regularly, available on the worldwide web at sanger.ac.uk/Software/Rfam/mirna/help/summary.shtml. This data base currently lists miRNAs from a number of organisms, exemplified in Table 1.

TABLE 1
A list of organisms and the number of miRNAs known
as listed in miRNA Registry on Jun. 5, 2006.

| | Organism | miRNA Number |
|---|---|---|
| Arthropoda | Anopheles gambiae | 38 |
| | Apis mellifera | 25 |
| | Drosophila melanogaster | 78 |
| | Drosophila pseudoobscura | 73 |
| | Anopheles gambiae | 38 |
| | Apis mellifera | 25 |
| Nematoda | Caenorhabditis briggsae | 79 |
| | Caenorhabditis elegans | 114 |
| Aves | Gallus gallus | 122 |
| Mammalia | Homo sapiens | 227 |
| | Mus musculus | 230 |
| | Rattus norvegicus | 191 |
| | Canis familiaris | 6 |
| | Ovis aries | 4 |
| Amphibia | Xenopus laevis | 7 |
| Primates | Ateles geoffroyi | 45 |
| | Gorilla gorilla | 86 |
| | Homo sapiens | 326 |
| | Lagothrix lagotricha | 48 |
| | Lemur catta | 16 |
| | Macaca mulatta | 71 |
| | Macaca nemestrina | 75 |
| | Pan paniscus | 89 |
| | Pan troglodytes | 83 |
| | Pongo pygmaeus | 84 |
| | Saguinus labiatus | 42 |
| Rodentia | Mus musculus | 249 |
| | Rattus norvegicus | 195 |
| Pisces | Danio rerio | 369 |
| | Fugu rubripes | 131 |
| | Tetraodon nigroviridis | 131 |
| Viridiplantae | Arabidopsis thaliana | 117 |
| | Glycine max | 22 |
| | Medicago truncatula | 16 |
| | Oryza sativa | 177 |
| | Populus trichocarpa | 213 |
| | Saccharum officinarum | 16 |
| | Sorghum bicolor | 64 |
| | Zea mays | 40 |
| Viruses | Epstein Barr virus | 5 |
| | Kaposi sarcoma-associated | 11 |
| | herpesvirus | |
| | Human cytomegalovirus | 9 |
| | Mouse gammaherpesvirus 68 | 9 |

In one embodiment, one or more miRNA sequences from one or more organisms are amplified, measured and detected, simultaneously or sequentially, using the primers and methods of the invention.

### EXAMPLES

### Example 1

This example illustrates that the attachment of a flap sequence (non-complementary to the target sequence) to both primers improve amplification efficiency substantially. Amplification efficiency improves as the flap sequence length is increased to 12 bases. Little or no improvement was observed with sequences longer than 12 base pairs. It was also shown that flap sequences in both primers are preferred over primer pairs where only one primer contains a flap sequence.

Materials and Methods

1.1 Oligonucleotides

PCR primers were synthesized using standard phosphoramidite chemistry. The MGB-FL-5′-ODN-Q probes were prepared by automated DNA synthesis on a minor groove binder modified polystyrene support using 5′-β-cyanoethyl phosphoramidites (Glen Research, VA) designed for synthesis of oligonucleotide segments in 5′→3′ direction as described in U.S. application Ser. No. 10/227,001, hereby incorporated herein by reference in its entirety for all purposes. Oligonucleotide synthesis was performed on an ABI 3900 synthesizer according to the protocol supplied by the manufacturer using a 0.02M iodine solution. Modified base phosphoramidites were synthesized based on methods previously disclosed (WO 03/022859 and WO 01/64958). The MGB Eclipse Probes were synthesized as described previously (Afonina et al, Biotechniques, 32, 940-949 (2002)). 6-Carboxyfluorescein (FAM), Yakima Yellow™ reporting dyes were introduced at the last step of the MB Eclipse probe synthesis using the corresponding phosphoramidites (Glen Research, Sterling, Va.). A fluorescein phosphoramidite described in U.S. application Ser. No. 10/227,001 was used. All oligonucleotides were purified by reverse phase HPLC. The sequences of the oligonucleotides used in Example 1 are shown in Table 1.

TABLE 2

Primer and Probe Sequences

| | | | |
|---|---|---|---|
| # | ODN Type | Sequence 5′-3′ | Length |

| | | | |
|---|---|---|---|
| 1 | Eclipse | MGB-Q-AGTAGA*TGTGGA*TGA- | 15 |
| | Probe | FAM | |
| 2 | Pleides | MGB-FAM-G*AAG*TAG*A*TG*TG* | 16 |
| | Probe | G*ATG*-Q | |
| 3_{F-12} | Sense | aataaatcataagcctttctttcatt | 30 |
| | Primer | ccctctc | |
| 3_{F-9} | Sense | aaatcataagcctttctttcattccc | 27 |
| | Primer | tctc | |
| | | atatttgatccacacacgttggtc | |
| 3_{F-6} | Sense | tcataagcctttctttcattccctct | 24 |
| | Primer | c | |
| 3_{F-3} | Sense | taagcctttctttcattccctctc | 21 |
| | Primer | | |
| 3_{F-0} | Sense | gcctttctttcattccctctc | 18 |
| | Primer | | |
| 4_{F-12} | Anti-sense | aataaatcataactgaagcaacatcc | 31 |
| | Primer | tgacagtc | |
| 4_{F-9} | Anti-sense | aaatcataactgaagcaacatcctga | 28 |
| | Primer | cagtc | |
| 4_{F-6} | Anti-sense | tcataactgaagcaacatcctgacag | 25 |
| | Primer | tc | |
| 4_{F-3} | Anti-sense | taactgaagcaacatcctgacagtc | 22 |
| | Primer | | |
| 4_{F-0} | Anti-sense | ctgaagcaacatcctgacagtc | 19 |
| | Primer | | |

The MGB ligand is DPI₃. Q is Eclipse Dark Quencher. G* is PPG; A* is Super A. F designate a Flap and the number following indicates the length of the flap sequence. The bold underlined sequence represents the flap sequence. FAM is fluorescein

1.2 Tₘ Prediction

MGB Eclipse™ Design Software (Epoch Biosciences, Bothell, Wash.) was used to design probe and primers. One of the features of the software is the ability to design primers or probes containing more than three consecutive Gs, known to be poor detection probes due to G:G self-association, and indicating an appropriate substitution of G with PPG. Additionally, the software can now design probes that incorporate Super A and Super T modified bases in AT-rich sequences to improve duplex stability.

1.3 Real-Time PCR

Real time PCR was conducted either in an ABI Prism® 7900 instrument (Applied Biosystems, Foster City, Calif.). Fifty cycles of three step PCR (95° C. for 5 s, 56° C. for 20 s and 76° C. for 20 s) after 2 min at 50° C. and 2 min at 95° C. were performed. The reactions contained 0.2 μM MGB-FL-ODN-Q or MGB Eclipse™ probe, 100 nM primer complementary to the same strand as the probe, 1 μM opposite strand primer, 125 μM dATP, 125 μM dCTP, 125 μM TTP, 250 μM dUTP, 0.25 U JumpStart DNA polymerase (Sigma), 0.15 U of AmpErase Uracil N-glycosylase (Applied Biosystems) in 1× PCR buffer (20 mM Tris-HCl pH 8.7, 40 mM NaCl, 5 mM MgCl₂) in a 15 μL reaction. The increase in fluorescent signal was recorded during the annealing step of the reaction.

1.4 The Effect of Flap Length in a Primer on Amplified Signal

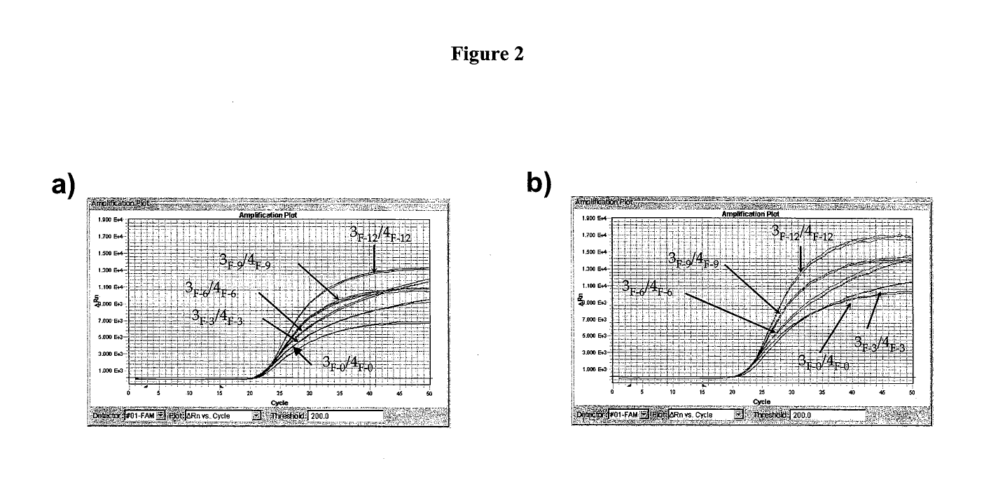

FIG. 2 demonstrates the effect of flap length on amplification efficiency. The detection of the amplified target with a MGB Eclipse probe and Pleiades probe is shown in FIGS. 2a) and b), respectively. As shown the primer pair with 12-mer flaps produced the greatest signal regardless of the probe type.

1.5 The Effect of the Presence of a Flap in One or Both Primers on Amplified Signal

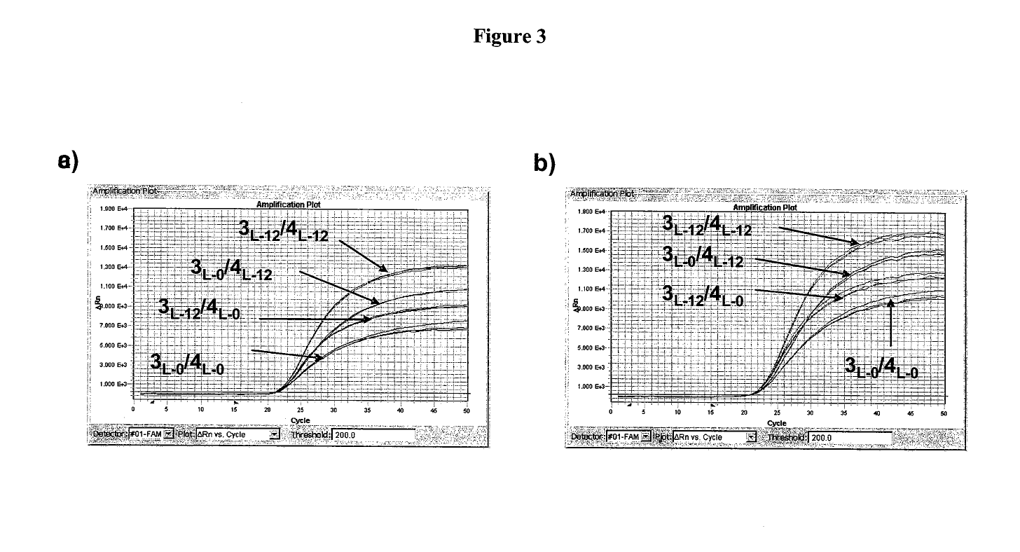

FIGS. 3a) and b) demonstrates that a primer pair with 12-mer flaps provide better amplified signal than amplification where only one primer contains a 12-mer flap. Amplified target is detected with MGB Eclipse probe (MGB-Q-ODN-FL) and Pleiades probe (MGB-FL-ODN-Q) in FIGS. 3a) and 3b), respectively.

### Example 2

This example illustrates that amplification with primers containing a 12-mer flap in both primers is more efficient than amplification with a flap in only one primer.

PCR amplification is performed as described in Example 1. The amplified target is detected either a MGB-Q-ODN-FL (MGB Eclipse Probe) or a MGB-FL-ODN-Q (Pleiades Probe). The primer, probe and amplified target sequences are shown for the MGB Eclipse and Pleiades assays are shown in Table 3.

TABLE 3

Primer and Probe Sequences

| | | | |
|---|---|---|---|
| # | ODN Type | Sequence 5′-3′ | Length |

| | | | |
|---|---|---|---|
| 5 | Sense | atatttgatccacacacgttggtc | 24 |
| | Primer | | |
| 5_{F-12} | Sense Flap | aataaatcataaatatttgatccaca | 36 |
| | Primer | cacgttggtc | |
| 6 | Anti-sense | aaggtatttgagcggcttcctc | 22 |
| | Primer | | |
| 6_{F-12} | Anti-sense | aataaatcataaaaggtatttgagcg | 34 |
| | Flap Primer | gcttcctc | |
| 7 | Eclipse | MGB-Q-GCAGAAAA*CAAAACAGG- | 17 |
| | Probe | FAM | |
| 8 | Pleiades | MGB-FAM-G*TTTTAACCG*TG*CTG | 18 |
| | | *AG*C-Q | |
| | Probe | | |

Sequence of the amplicon target in the MGB Eclipse assay aataaatcataaatatttgatccacacacgttggtcTTTTAACCGTGCTGA [symbol] TTAAGAAGAGCCGGGTGGCAGCTGACAgaggaagccgctcaaataccttttatgatttatt
aataaatcataaatatttgatccacacacgttggt [symbol] AGAAAACAAAACAGGTTAAGAAGAGCCGGGTGGCAGCTGACAgaggaagccgctcaaataccttttatgatttatt
The MGB ligand is DPI₃. Q is Eclipse Dark Quencher. G* is PPG; A* is Super A. F designate a Flap and the number following indicates the length of the flap sequence. FAM is fluorescein. The primers are shown in bold lower case with the flap sequence underlined. The probe sequence is shown upper case italics. The probe overlaps with one base with the sense primer. The Pleiades probe overlaps with one base with the sense primer.

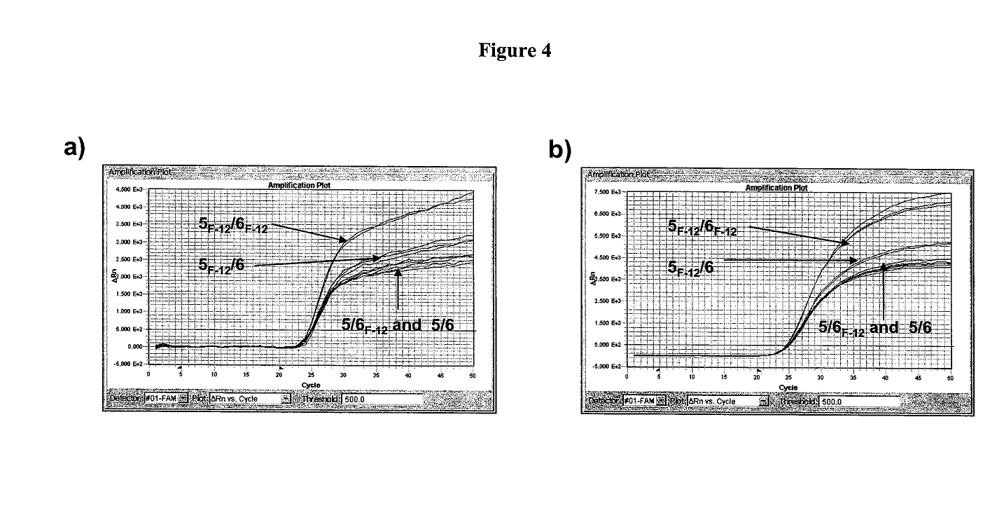

As shown in FIG. 4 a primer pair (5F-12/6F-12) with two 12 base flaps showed improved real-time amplification over primer pairs with one 5F-12/6 or no flap (5/6) present.

### Example 3

This example further illustrates that that amplification with primers containing a 12-mer flap in both primers is more efficient than amplification with a flap in only one primer.

PCR is performed as described in example 1. The amplified target is detected either a MGB-Q-ODN-FL (MGB Eclipse Probe) or a MGB-FL-ODN-Q (Pleiades Probe) conjugate. The primer, probe and amplified target sequences are shown in Table 4.

TABLE 4

Primer and Probe Sequences

| | | |
|---|---|---|
| ODN Name | sequence 5′-3′ | Length |

| | | | |
|---|---|---|---|
| 9 | Sense | gcctttctttcattccctctc | 21 |
| | Primer | | |
| 9_{F-12} | Sense Flap | aataaatcataagcctttctttcatt | 33 |
| | Primer | ccctctc | |
| 10 | Anti-sense | ctgaagcaacatcctgacagtc | 22 |
| | Primer | | |
| 10_{F-12} | Anti-sense | aataaatcataactgaagcaacatcc | 34 |
| | Flap Primer | tgacagtc | |
| 11 | Eclipse | MGB-Q-AGTAGA*TGTGGA*TGA- | 15 |
| | Probe | FAM | |
| 12 | Pleiades | MGB-FAM-G*AAG*TAG*A*TG*TG* | 16 |
| | Probe | G*ATG*-Q | |

Amplified Target
aataaatcataagcctttctttcattccctctcTGAAAAGTATTCCAACGTGATATTCCTTGAAGTAGATGTGGATgactgtcaggatgttgcttcagttatgatttatt
The MGB ligand is DPI₃. Q is Eclipse Dark Quencher. G* is PPG; A* is Super A. F designate a Flap and the number following indicates the length of the flap sequence. The primers are shown in bold lower case with the flap sequence underlined. FAM is fluorescein

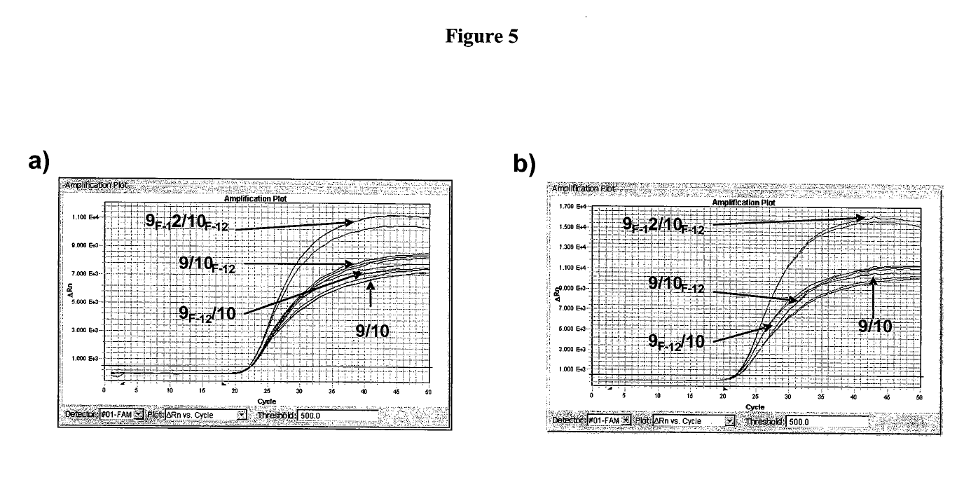

As shown in FIG. 5 a primer pair (9F-12/10F-12) with two 12 base flaps showed improved real-time amplification over primer pairs with one 9F-12/10, 9/_{10F-12} or no flap (9/10) present.

### Example 4

This example illustrates the improvement observed with flap primers in RT-PCR. It also illustrates the ability of modified bases and flap primers to improve amplification.

The one tube reaction was performed using QIAGEN (Valencia, Calif.) One-Step RT-PCR Kit with different amounts of human parainfluenza RNA (10⁵ to 10⁰ copies) per reaction. We followed the protocol suggested by a manufacturer with minor exceptions. Instead of recommended 1.0 μM of each gene-specific primer. Pleiades probe was added to a final concentration of 0.2 μM to enable real time detection. RNase inhibitor (Ambion, Austin, Tex.) was used at 15 units per reaction. Thermal cycler conditions were within recommended range and included 30 min at 60° C. for reverse transcription, 15 min at 95° C. for the initial PCR activation step, and 50 3-step cycles of denaturation (50 sec at 95° C.), annealing (20 sec at 56° C.), extension (20 sec at 76° C.). Fluorescent readings were taken at the annealing stage of PCR.

The primer, probe and target amplicon sequences are shown in Table 5.

TABLE 5

Primer and Probe Sequences

| | |
|---|---|
| ODN | Probe |

| | | | | |
|---|---|---|---|---|
| # | Type | Sequence 5′-3′ | Tₘ ° C. | Length |

| | | | | |
|---|---|---|---|---|
| 13 | Primer | taatgaaggtagtctaacaca | 64.8 | 26 |
| | | tcctg | | |
| 14_{F-0} | SPrimer | gtagtctaacacatcctg | 59.3 | 18 |
| 14_{F-12} | FPrimer | aataaatcataagtagtctaa | 63.7 | 30 |
| | | cacatcctg | | |
| 15_{F-0} | SPrimer | gtagtcta*acacatcctg | 61.3 | 18 |
| 15_{F-12} | Fprimer | aataaatcataagtagtcta* | 64.9 | 30 |
| | | acacatcctg | | |
| 16_{F-0} | SPrimer | gtagtcta*acaca*tcctg | 64.5 | 18 |
| 16_{F-12} | Fprimer | aataaatcataagtagtcta* | 66.9 | 30 |
| | | acaca*tcctg | | |
| 17 | Primer | ccccaatatctcattattacc | 69.0 | 32 |
| | | tggaccaagtc | | |
| 18_{F-0} | SPrimer | attacctggaccaagtct | 61.5 | 18 |
| 18_{F-12} | FPrimer | aataaatcataaattacctgg | 65.0 | 30 |
| | | accaagtct | | |
| 19_{F-0} | SPrimer | attacctgga*ccaagtct | 64.8 | 18 |
| 19_{F-12} | Fprimer | aataaatcataaattacctgg | 66.9 | 30 |
| | | a*ccaagtct | | |
| 20 | Probe | MGB-Q-G*TTGATCCAGA*AA | 67.4 | 17 |
| | | GTA*G-FAM | | |
| 21 | Flap | aataaatcataa | 31.3 | 12 |

Human parainfluenza RNA Amplicon (82 bp; 13/17 primer)
TAATGAAGGTAGTCTAACACATCCTGAGATTGTGGTTGATCCAGAAAGTAGACTTGGTCCAGGTAATAATGAGATATTGGGG
The MGB ligand is DPI₃. Q is Eclipse Dark Quencher. G* is PPG. A* is Super A. F designate a Flap and the number following indicates the length of the flap sequence. The bold underlined sequence represents the flap sequence. FAM is fluorescein

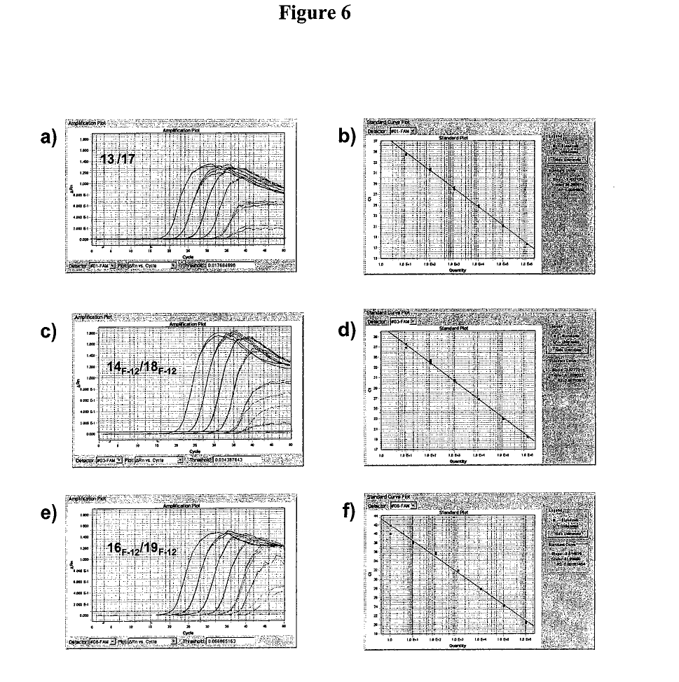

The non-flap primer pair (13/17) with calculated Tₘs of 64.0° C. and 69.0° C., respectively showed a 6 log titration curve (FIG. 6a) and b)). Primers 13 and 17 were shortened 8 and 15 bases, respectively and a 12 base flap was added to each of these shortened primers. The resulting Tₘs of probes 14_{F-12} and 18_{F-12} are 63.7 and 65.0° C., as shown in FIG. 6c) the signal is about 25% higher than the non-flap primers shown in FIG. 6a). FIG. 6d) still shows a 6 log titration curve with these two flap primers. When on or two As were substituted with Super A in these two flap primers the resulting 16_{F-12} and 19 _{F-12} primer pair showed 63.7 and 65.0° C. Tₘs, respectively, This primer pair increased the amplification signal to detect amplified target over 7 logs as shown in FIG. 6e) and f).

### Example 5

This example illustrates that ability of flap primers to improve amplification detection in both singleplex and biplex assays used in gene expression assays. In this case beta-2-microglobulin (B2MG) was used as a housekeeping gene and biplexed with a gene of interest (GI), Homo sapiens mitogen-activated protein kinase 3 (MAP2K3) gene.

The primer and probe sequence of B2MG and GI are shown in Table 6. PCR was performed as described in Example 1, with the exception that the primer concentrations for the B2MG amplification was 1 μM for the excess primer and 0.040 μM for the limiting primer.

TABLE 6

Primer and Probe Sequences Biplex
Amplification

ODN

| | | | |
|---|---|---|---|
| # | Type | Sequence 5′-3′ | Length |

| | | | |
|---|---|---|---|
| | | Beta-2-microglobulin (B2MG) | |
| 22 | Primer | gcctgccgtgtga*acca*tgtga*ctttgt | 29 |
| | | c | |
| 23 | Primer | cggcatcttcaaacctccatga | 22 |
| 22_{F-12} | Flap | aataaatcataagcctgccgtgtga*acca* | 41 |
| | Primer | tgtga*ctttgtc | |
| 23_{F-12} | Flap | aataaatcataacggcatcttcaaacctcca | 34 |
| | Primer | tga | |
| 24 | Probe | MGB-Q-GTTAAGTGGGATCGAGA-FAM | 17 |
| | | Gene of Interest (GI) | |
| 25 | Primer | CAATTCCAGAGGACATCCTTGG | 22 |
| 26 | Primer | TAAGGACATTGGAGGGCTTCACATC | 25 |
| 25_{F-12} | Flap | AATAAATCATAACAATTCCAGAGGACATCCT | 34 |
| | Primer | TGG | |
| 26_{F-12} | Flap | AATAAATCATAATAAGGACATTGGAGGGCTT | 37 |
| | Primer | CACATC | |
| 27 | Probe | MGB-Q-CTGTGTCTATCGTGCGG-TET | 17 |

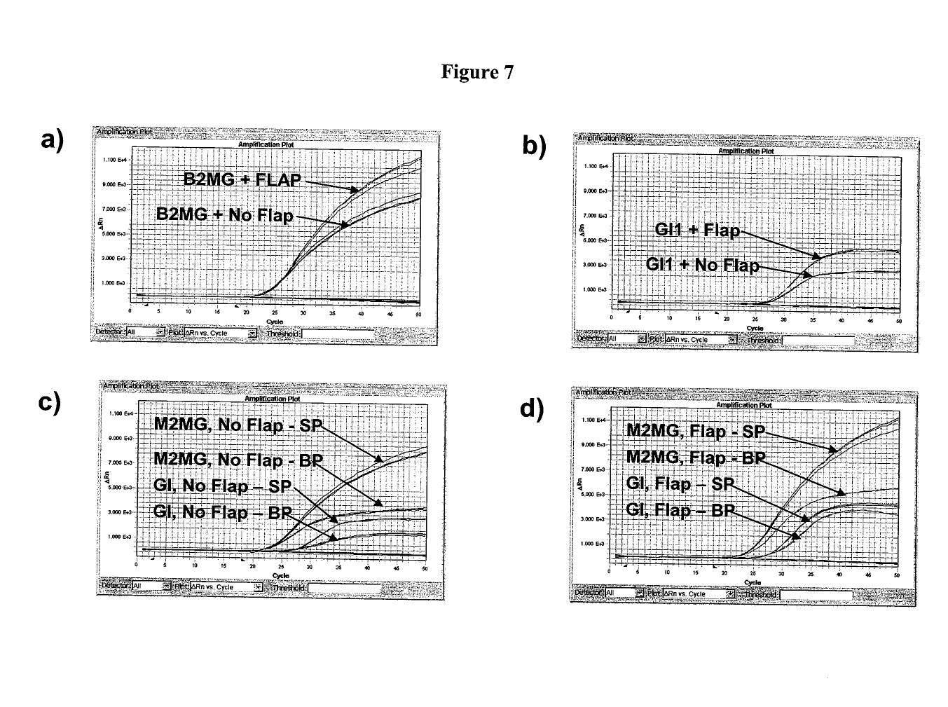

As shown in FIG. 7a) the introduction of a 12 base flap in the singleplex amplification of B2MG shows improved amplification. Similarly the singleplex amplification of GI of interest benefited from the introduction of flap primers (FIG. 7b)). Comparison of FIGS. 7c) and d) indicate that even when the amplification of B2MG and the GI are biplexed, improved amplification is seen with the flap primers.

### Example 6

This example illustrates the amplification of short targets with flap primers and particularly of the DNA or the cDNA of miRNA hsa-miR-139 target. This miRNA is 18 bases long. The primer, probe and miRNA sequences are shown in Table 7.

TABLE 7

The primer and target sequences used for
the detection of hsa-miR-139 DNA target

ODN

| | | | |
|---|---|---|---|
| # | Type | Sequence 5′-3′ | Tₘ |

| | | | |
|---|---|---|---|
| 22 | Primer | tctacagtgc | 41.8° C. |
| 22_{F-12} | Flap | aataaatcataatctacagtgc | 57.8° C. |
| | Primer | | |
| 23 | Primer | t*ct*a*ca*gt*gc | 58.3° C. |
| 24 | Primer | agacacgtgc | 49.4° C. |
| 24_{F-12} | Flap | aataaatcataaagacacgtgc | 60.8° C. |
| | Primer | | |
| 25 | Primer | A*GA*CA*CGTGC | 63.0° C. |

Target sequence TCTACAGTGCACGTGTCT

The real-time PCR was performed as described in Example 1 with a target concentration of 1×10⁷ copies, with the only difference that the amplified target was detected with Sybr Green (Sigma-Aldrich, St. Louis, Mo.), using the manufactures protocol. Post amplification melt curve analysis was performed on an ABI Prism® instrument according to manufacturer's instructions with a temperature gradient ramp rate of 10%.

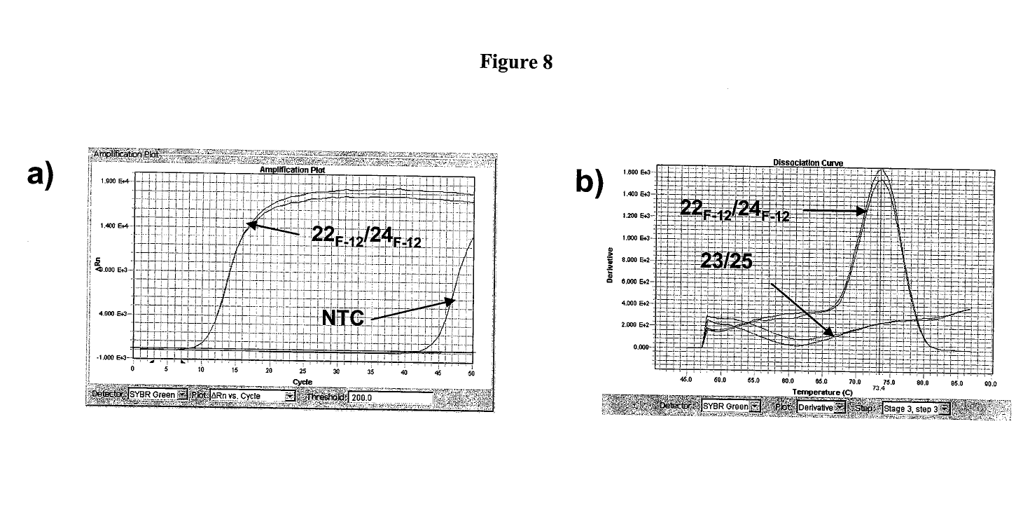

No amplification and detection of the 18-mer miRNA target with the primer pair 22/24 or when modified with Super A and T in the primer pair 23/24 was observed, data not shown. The primer pair 22F-12/24F-12 showed amplification (FIG. 8a)) which was confirmed in the melt curve analysis, data shown in FIG. 8b) where a melt curve with the expected Tₘ was observed while amplification with primer pair (23/25) showed no product in the melt curve analysis. The no template control showed some amplification signal with a Cₜ of about 44 cycles. It is anticipated that when concentrations lower than 1×10⁷ copies are analyzed that amplification of NTC will be small and with Cₜs greater than 40 cycles, the number of cycles typically analyzed.

### Example 7

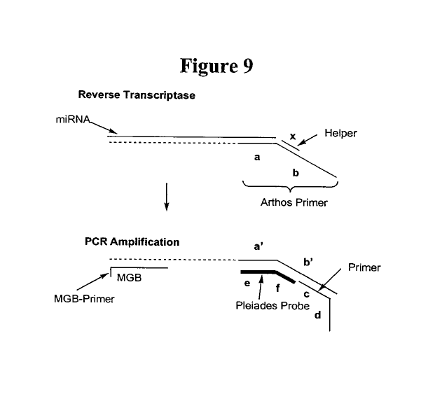

This example demonstrates amplification of target nucleic acids using the method set forth in FIG. 9, wherein a flap primer is used as the RT primer and a primer covalently attached to either a DPI₂ or DPI₃ minor groove binder is used as an amplification primer (i.e., PCR primer) to further amplify and detect hsa-miR-142-3P targets.

TABLE 8

Primer, probe, and target sequences used for
the detection of hsa-miR-142-3P RNA Target:
UGUAGUGUUUCCUACUUUAUGGA (23 bp).
Non-complementary sequences are underlined.

Oligo-

| | | | | |
|---|---|---|---|---|
| # | nucleotide | Sequence 5′-3′ | Tₘ ° C. | Length |

| | | | | |
|---|---|---|---|---|
| 1 | Limiting | DPI₂-TGTAGTGTTTCCTACT | 56 | 16 |
| | Primer | | | |
| 2 | Limiting | DPI₃-TGTAGTGTTTCCTACT | 58 | 16 |
| | Primer | | | |
| 3 | Excess | AATAAATCATAAGTGGACGGT | 67.7 | 28 |
| | Primer | CCGAGGT | | |
| 4 | RT Primer | GTGGACGGTCCGAGGTCTGGA | | 38 |
| | | TACGACTCCATAAAGTA | | |
| 4a | RT Primer | GTGGACGGTCCGAGGTCTGGA | | 36 |
| | | TACGACTCCATAAAG | | |
| 5 | Pleiades | TTTATGGA*GTCGTATCC | 68 | 17 |
| | probe | | | |

Reverse Transcription

Reverse transcription was performed using the CLONTECH Advantage RT-for-PCR Kit from TAKARA BIO. Each reaction had a final volume of 20 μL and contained the following at final concentration: 50 mM Tris-HCl pH 8.3, 75 mM KCl, 3 mM MgCl₂, dNTP Mix 0.5 mM each, RNase inhibitor 1 unit/μL, MMLV reverse transcriptase ≧200 units/μg RNA, 50 nm RT primer and 1 μL of the appropriate concentration of synthetic RNA template. Reaction mixtures were placed into 0.2 μL thin-walled PCR tubes, then into the MJ Research PTC-200 Thermal Cycler. Samples were held at 16° C. for 30 minutes, then 42° C. for 30 minutes, then 94° C. for 5 minutes.

Real-Time PCR

Real-time PCR was conducted using the ABI Prism® Real-Time (Applied Biosystems, Foster City, Calif.). Samples were held at 95° C. for 2 min, followed by forty cycles of three step PCR (95° C. for 5 s, 56° C. for 20 s and 76° C. for 20 s). Samples then underwent a melt profile of 95° C. for 15 s, 35° C. for 15 s, then 90° C. for 15 s. Reactions were set up using the MGB Eclipse™ PCR Reagent Kit for SNPs (Nanogen, Bothell, Wash.), which contains the following at final concentration: 20 mM Tris-HCl, pH 8.7, 40 mM NaCl, 2.5 mM MgCl₂, 0.5 M Betaine, 0.25 mM dUTP, 0.125 mM dATP, 0.125 mM dCTP, 0.125 mM dGTP, and Jumpstart Taq Polymerase 0.075 units/μL.

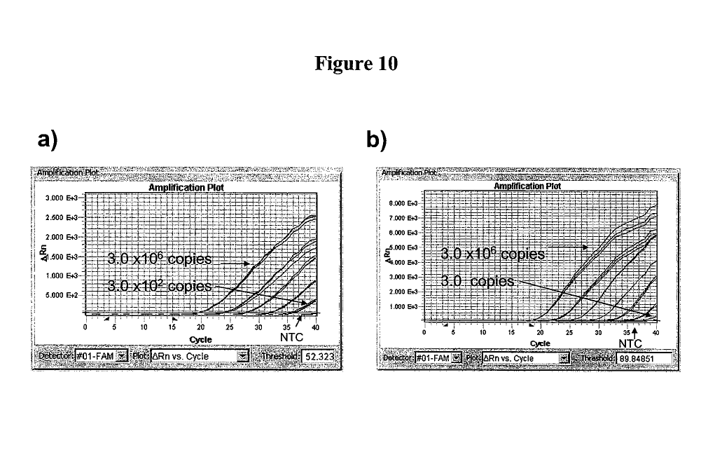

As shown in FIG. 10, both DPI₂- and DPI₃-coupled primers function satisfactorily in the detection of hsa-miR-142-3P RNA Target. FIG. 10 shows a) real-time amplification of titration of synthetic hsa-miR-142-3P target with limiting primer #1 containing a DPI₂ moiety attached to the 5′end and b) limiting primer #2 containing a DPI₃ moiety attached to the 5′end.

### Example 8

This example demonstrates amplification of target nucleic acids using the method set forth in FIG. 9, wherein a primer complex comprising a flap primer and a helper primer are used as the RT primer to amplify the target nucleic acid and a primer covalently attached to either DPI₃ minor groove binder is used as the amplification primer (i.e., PCR primer) to further amplify and detect to detect hsa-miR-142-3P targets. The sequence of the helper oligonucleotide is shown in Table 9.

TABLE 9

Helper sequences used for the detection
of hsa-miR-142-3P RNA Target:
UGUAGUGUUUCCUACUUUAUGGA (23 bp). A* is
Super A and T* is Super T.

| # | Oligonucleotide | Sequence 5′-3′ | Tₘ ° C. | Length |
|---|---|---|---|---|
| 6 | RT Helper Oligo | GT*CGTA*TCCA*GA | 52.3 | 12 |

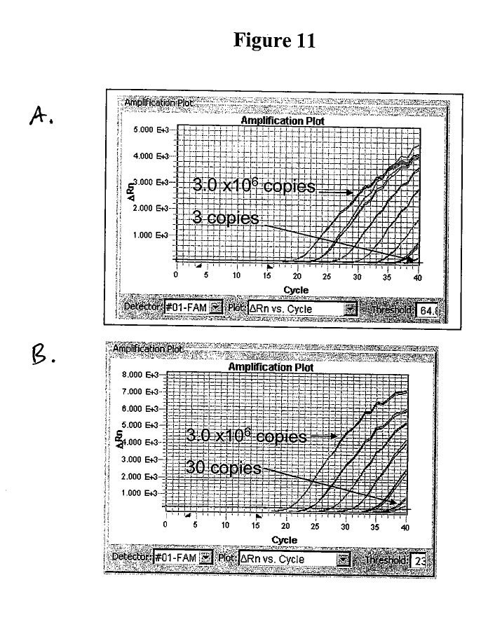

The reaction conditions set forth in Example 7 above were used with the following modification: the helper oligonucleotide is added to RT reaction mixture at a final concentration of 100 nM. As shown in FIG. 11 the synthetic hsa-miR-142-3P target with limiting primer #1 containing a DPI₃ moiety attached to the 5′end is amplified. FIG. 11A shows the results from real-time amplification of the synthetic hsa-miR-142-3P target using RT primer 4 and FIG. 11B shows the results from real-time amplification of the synthetic hsa-miR-142-3P target using RT primer 4a.

### Example 9

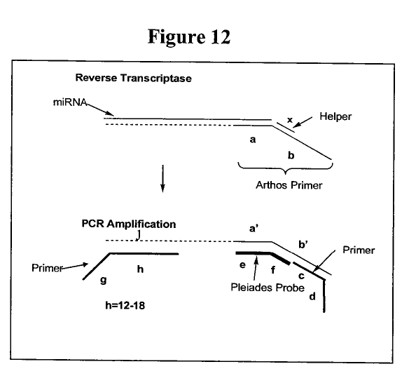

This example demonstrates amplification of a target nucleic acid sequence using the method set forth in FIG. 12, wherein a primer complex comprising a flap primer and a helper oligonucleotide is used as the RT primer and two flap primers are used as amplification primers (i.e., PCR primers) to further amplify and detect hsa-miR-142-3P targets, in the presence of helper oligonucleotide. The reaction conditions to run the RT and amplification (i.e., PCR) reactions were as described in Examples 7 and 8 above. The primer, probe, helper and target sequences are shown in Table 10.

TABLE 10

Primer, probe, helper and target
sequences used for the detection of
hsa-miR-142 3P target.
UGUAGUGUUUCCUACUUUAUGGA (23 bp). A* is
Super A and T* is Super T.
Non-complementary sequence is underlined.

Oligo-

| | | | | |
|---|---|---|---|---|
| # | nucleotide | Sequence 5′-3′ | Tₘ ° C. | Length |

| | | | | |
|---|---|---|---|---|
| 7 | Limiting | GCCCGCTCATAATGTAGTGTTT | 66.6 | 28 |
| | Primer | CCTACT | | |
| 3 | Excess | AATAAATCATAAGTGGACGGTC | 67.7 | 28 |
| | Primer | CGAGGT | | |
| 4 | RT Primer | GTGGACGGTCCGAGGTCTGGAT | | 36 |
| | | ACGACTCCATAAAG | | |
| 6 | RT Helper | GT*CGTA*TCCA*GA | 52.3 | 12 |
| | Primer | | | |
| 5 | Pleiades | TTTATGGA*GTCGTATCC | 68 | 17 |
| | Probe | | | |

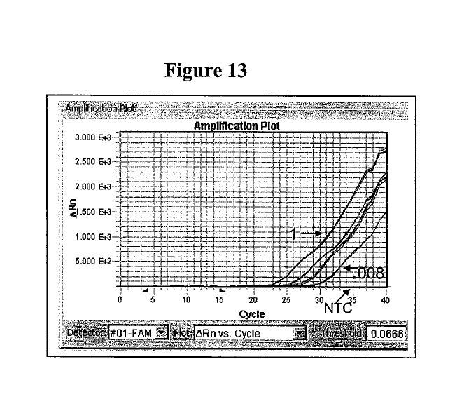

FIG. 13 shows a titration curve (5 fold dilutions) of hsa-miR-142-3P target.

### Example 10

This example demonstrates amplification of a target sequence (i.e., hsa-miR-142-3P targets) using the method set forth in FIG. 12, wherein a flap primer comprising an annealed helper oligonucleotide is used as the RT primer. The reaction conditions were as described in Examples 7 and 8 above. The limiting primer sequence is shown in Table 11.

TABLE 11

Limiting prime sequence of hsa-miR-142-3P
assay

| | | | | |
|---|---|---|---|---|
| # | Oligonucleotide | Sequence 5′-3′ | Tₘ ° C. | Length |

| | | | | |
|---|---|---|---|---|
| 8 | Limiting Primer | GACGGTCCGAGGTCTGGA | 68.2 | 19 |
| | | T | | |

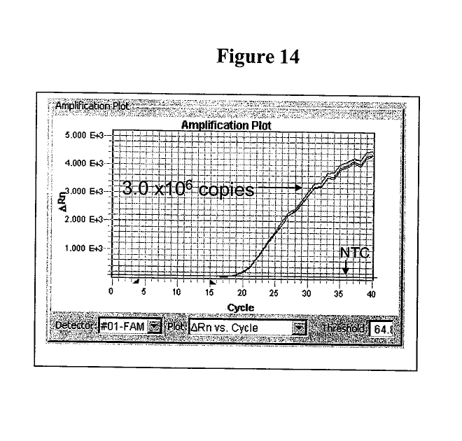

FIG. 14 shows the real-time amplification and detection of hsa-miR-142-3P target. Reverse transcription performed with HL-60 total RNA (Strategene, La Jolla, Calif.). Real time PCR was performed as in Example 9, except that limiting primer 7 was substituted with limiting primer 8.

### Example 11

This example demonstrates amplification of a target sequence (i.e., a synthetic hsa-miRNA16-1 precursor molecule) using the method set forth in FIG. 12, wherein a flap primer comprising an annealed helper oligonucleotide is used as the RT primer and real-time detection of the target nucleic acids. The reaction conditions for the RT and PCR reactions were as described in Examples 7 and 8 above, except that the concentrations of oligonucleotides 9, 10, 11, 6, and 2 were 250, 1500, 50, 100 and 200 nm, respectively. The primer, probe, helper and target sequences are shown in Table 12.

TABLE 12

Primer, probe, and target sequences used
for the detection of hsa-miR-16-1 DNA
target. UAGCAGCACGUAAAUAUUGGCGUUAAGAUUCUA
AAAUUAUCUCCAGUAUUAACUGUGCUGCUGAA (65 bp)
A* is Super A. T* is Super T. The primers
are shown with the flap sequence
underlined.

Oligo-

| | | | | |
|---|---|---|---|---|
| # | nucleotide | Sequence 5′-3′ | Tₘ ° C. | Length |

| | | | | |
|---|---|---|---|---|
| 9 | Sense | CCCGCTCATAATCT*CCAGTA | 65.0 | 25 |
| | Primer | *TTAAC | | |
| 10 | Antisense | CGCATTGATCAATCGTACGGT | 65.9 | 23 |
| | Primer | CT | | |
| 11 | RT Primer | CGCATTGATCAATCGTACGGT | | 40 |
| | | CTCTGGATACGACTCAGCA | | |
| 6 | RT Helper | GT*CGTA*TCCA*GA | 52.3 | 12 |
| 2 | Pleiades | TGAGT*CGTA*TCCA*GAG | 66.8 | 16 |
| | Probe | | | |

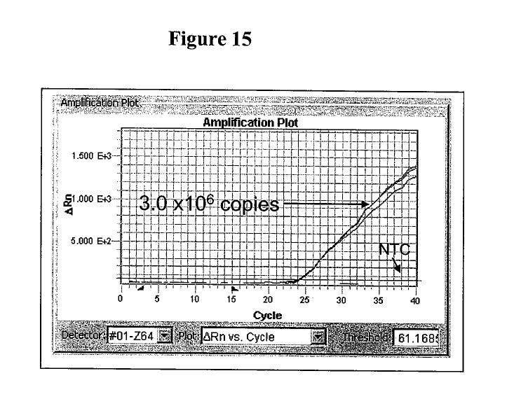

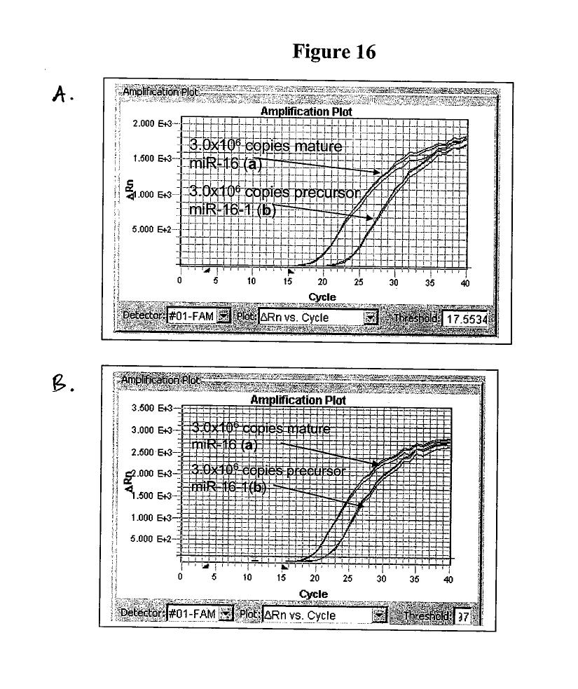

FIG. 15, shows the detection of hsa-miR-16-1 precursor target. As shown in FIG. 16 below, precursor miRNA can be distinguished from no template control.

### Example 12

This example demonstrates real-time amplification a) mature hsa-miRNA-16 and b) of hsa-miR-16 mature sequence from hsa-miR-16-1 precursor molecule. The reaction conditions for the RT and PCR reactions were as described in Examples 7 and 8 above, except that the concentrations of oligonucleotides 13, 14, 15, 6, and 16 were 1500, 250, 50, 100 and 200 nm, respectively. The primer, probe, helper and target sequences are shown in Table 13.

TABLE 13

Primer, probe, and target sequences used
for the detection of hsa-miR-16 target.
t: UAGCAGCACGUAAAUAUUGGCG (22 bp). A* is
Super A. G* is Super G. The primers are
shown with the flap sequence underlined

Oligo-

| | | | | |
|---|---|---|---|---|
| # | nucleotide | Sequence 5′-3′ | Tₘ ° C. | Length |

| | | | | |
|---|---|---|---|---|
| 13 | Sense | CCCGCTCATAATAGCAGCA | 69.1 | 23 |
| | Primer | CGTA | | |
| 14 | Antisense | AATAAATCATAAGTGGACG | 68.1 | 28 |
| | Primer | GTCCGAGGT | | |
| 15 | RT Prime | GTGGACGGTCCGAGGTCTG | | |
| | | GATACGACCGCCAA | | |
| 15 a | RT Primer | GTGGACGGTCCGAGGTCTG | | 38 |
| | | GATACGACCGCCAATATTT | | |
| 6 | RT Helper | GT*CGTA*TCCA*GA | 52.3 | 12 |
| 16 | Pleiades | G*CCAA*TA*TTTA*CGTG | 66.8 | 17 |
| | Probe | CT | | |

FIG. 16A shows the results from real-time amplification of a) mature hsa-miR-16 and b) hsa-miR-16 mature sequence from hsa-miR-16-1 precursor molecule using primer #15. FIG. 16B shows the results from real-time amplification of a) mature hsa-miR-16 and b) hsa-miR-16 mature sequence from hsa-miR-16-1 precursor molecule using primer #15a. As shown in FIG. 16, the assay can be used to differentiate mature target sequences from precursor molecules.

### Example 13

This example demonstrates the ability of the let-7a specific real-time amplification assay to discriminate between let-7a, b, c, d, e synthetic miRNA templates. The reaction conditions for the RT and PCR reactions were as described in Examples 7 and 8 above, except that the concentrations of oligonucleotides 17, 18, 19, and 20 were 1500, 2500, 50 and 200 nm, respectively. The primer, probe and target sequences for let-7a are shown in Table 14.

TABLE 14

Oligo-

| | | | | |
|---|---|---|---|---|
| # | nucleotide | Sequence 5′-3′ | Tₘ ° C. | Length |

| | | | | |
|---|---|---|---|---|
| 17 | Sense | CCCGCTCATAATGAGGTAGTA | 66.6 | 24 |
| | Primer | GGT | | |
| 18 | Antisense | AATAAATCATAAGTGGACGGT | 67.6 | 28 |
| | Primer | CCGAGGT | | |
| 19 | RT Primer | GTGGACGGTCCGAGGTCTGGA | | 38 |
| | | TACGACAACTATACAAC | | |
| 20 | Pleiades | TGGATACGACA*ACTA*TA*C | 67.2 | 18 |
| | Probe | | | |

The ability of the let-7a assay to discriminate the closely related let-7b, c, d and e synthetic templates is illustrated in Table 15.

TABLE 15
Cₜ and ΔCₜ Values for the let-7a assay with
let-7a, b, c, d and e synthetic templates

| | | Synthetic miRNA target | | | | | |
|---|---|---|---|---|---|---|---|
| | | let-7a | let-7b | let-7c | let-7d | let-7e | |
| miRNA | let-7a | 16.4 | 21.9 | 17.0 | 22.5 | 26.8 | Ct |
| assays | let-7a | 0.0 | 5.5 | 0.6 | 6.1 | 10.4 | ΔCt |

ΔCₜ is the difference in Cₜ between the indicated miRNA targets

### Example 14

This example demonstrates the differentiation of the closely related let-7a and let-7-d by melting curve analysis. The assay conditions of Example 7 were used except that RT-primer 19 was substituted with a RT-primer having the following sequence: GTGGACGGTCCGAGGTCTGGATACGACAACTAT and the method to distinguish targets with one or more mismatches by melting curve analysis with hybridization-bases assays was the method disclosed in U.S. Patent Publication No. 20030175728 which is expressly incorporated herein by reference in its entirety. The let-7a assay reagents were used as described in Example 13 above to perform the amplification and to generate the melting curves for the synthetic let-7a and let-d targets. The sequences of let7a and let-7d are shown below:

| | |
|---|---|
| Let-7a | ugagguaguagguuguauaguu. |
| Let-7d | agagguaguagguugcauagu |

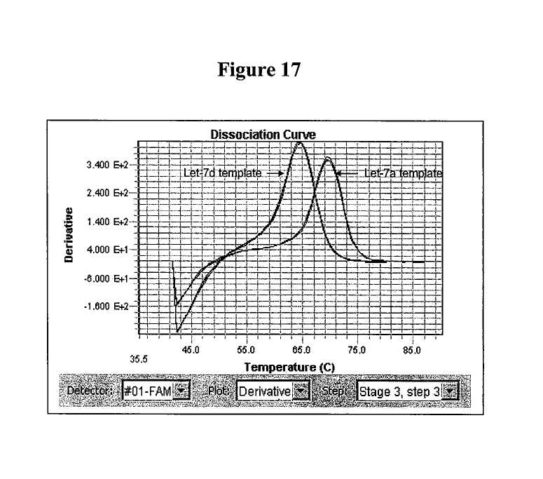

The two mismatches (in bold) and the one base deletion in let-7d in relation to let-7a results in a hybrid with the let-7a probe of lower stability as reflected by the melting curves illustrated in FIG. 17. The melting curves of let-7a and let-7d amplicon templates probed with let-7a probe.

### Example 15

This example demonstrates the amplification and detection of a miRNA target with a MGB-Fl-oligonucleotide primer. The reaction conditions for the RT and PCR reactions were as described in Examples 7 and 8, above. The primer, probe and target sequences are shown in Table 16.

TABLE 16

Primer and probe sequences used for the
detection of hsa-miR-16 RNA Target:
UGUAGUGUUUCCUACUUUAUGGA (23 bp)

Oligo-

| | | | | |
|---|---|---|---|---|
| # | nucleotide | Sequence 5′-3′ | Tₘ ° C. | Length |

| | | | | |
|---|---|---|---|---|
| 21 | Sense | AATAAATCATAAGCA*GCACG | 58.4 | 20 |
| | Primer | | | |
| 22 | Antisense | DPI₂-(FAM)-AATAAATCAT | 66.1 | 21 |
| | Labeled | AACGCCA*AT*AT | | |
| | Primer | | | |
| 23 | RT Primer | AATAAATCATAACGCCA*ATA | 55.5 | 24 |
| | | TT*T*A | | |

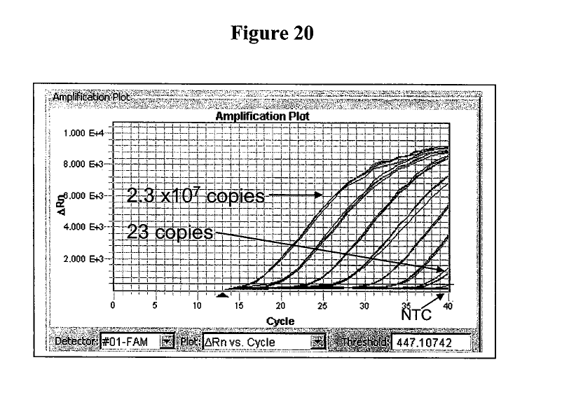

FIG. 20 shows a titration curve (10 fold dilutions) of hsa-miR-16 target with detection of 2.3×10⁷ to 23 copies.

### Example 16

This example demonstrates the biplex of the primers and probes for hsa-miR-16 and hsa-miR-21 assays with primers and probes for 18S RNA internal control assay.

The primer, probe, helper and target sequences for hsa-miR-16 and hsa-miR-21 are shown in Table 17 and Table 18, respectively. The primers and probe for 18S rRNA assay are included in Table 17. The concentration of miR-16 and miR-21 was determined relative to the concentration 18S rRNA in HL-60 total RNA.

The reaction conditions for the RT and PCR reactions were as described in Examples 7 and 8, except that the concentrations of in the RT reaction of oligonucleotides 26, 35, 32 and 6 were 50, 50, 1500 and 100 nm, respectively. In the PCR reaction the concentrations of oligonucleotides 24, 25, 29 and 30 were 250, 1500, 60 and 600 nM, respectively.

TABLE 17

Primer, probe, and target sequences used
for the detection of hsa miR-16 target
(5′-UAGCAGCACGUAAAUAUUGGCG-3′) and 18sRNA
target. A* is Super A. T* is Super T. G*
is Super G. The primers are shown with
the non-complementary sequences
underlined.

Oligo-

| | | | | |
|---|---|---|---|---|
| # | nucleotide | Sequence 5′-3′ | Tₘ ° C. | Length |

| | | | | |
|---|---|---|---|---|
| 24 | miR-16 | CCCGCTCATAATAGCAGCACG | 65 | 23 |
| | Sense | TA | | |
| | Primer | | | |
| 25 | miR-16 | AATAAATCATAAGTGGACGGT | 66 | 28 |
| | Antisense | CCGAGGT | | |
| | Primer | | | |
| 26 | miR-16 RT | GTGGACGGTCCGAGGTCTGGA | | 33 |
| | Primer | TACGACCGCCAA | | |
| 27 | miR-16 | AAATATTGGCGGTCGT | 69.9 | 16 |
| | Pleiades | | | |
| | Probe | | | |
| 6 | Helper | GT*CGTA*TCCA*GA | 52.3 | 12 |
| | Oligo | | | |
| 29 | 18S Sense | AATAAATCATAAGA*CACGGA | | 32 |
| | Primer | *CAGGA*TTGACAG | | |
| 30 | 18S | AATAAATCATAAAATTAA*CC | 62.8 | 30 |
| | Antisense | AGA*CAAATCG | | |
| | Primer/RT | | | |
| | Primer | | | |
| 31 | 18S | A*TTCTTA*GTTGGTGGAGC | 70 | 18 |
| | Pleiades | | | |
| | Probe | | | |

TABLE 18

Primer, probe, helper and target
sequences used for the detection of hsa
miR-21 (UAGCUUAUCAGACUGAUGUUGA) and 18S
rRNA targets. A* is Super A. T* is Super
T. The primers are shown with the
non-complementary sequences underlined.

Oligo-

| | | | | |
|---|---|---|---|---|
| # | nucleotide | Sequence 5′-3′ | Tₘ ° C. | Length |

| | | | | |
|---|---|---|---|---|
| 32 | miR-21 | GCCCGCTAGCTTATCAGACTG | 67.9 | 24 |
| | Sense | ATG | | |
| | Primer | | | |
| 32 | miR-21 | AATAAATCATAAGTGGACGGT | 66 | 28 |
| | Antisense | CCGAGGT | | |
| | Primer | | | |
| 34 | miR-21 | G*GATACGA*CTCAACA*TC | 69 | 17 |
| | Pleiades | | | |
| | Probe | | | |
| 35 | miR-21 RT | GTGGACGGTCCGAGGTCTGGA | | 33 |
| | Primer | TACGACTCAACA | | |
| 6 | Helper | GT*CGTA*TCCA*GA | 52.3 | 12 |
| | Oligo | | | |
| 29 | 18S Sense | AATAAATCATAAGA*CACGGA | | 32 |
| | Primer | *CAGGA*TTGACAG | | |
| 30 | 18S | AATAAATCATAAAATTAA*CC | 62.8 | 30 |
| | Antisense | AGA*CAAATCG | | |
| | Primer/RT | | | |
| | Primer | | | |
| 31 | 18S | A*TTCTTA*GTTGGTGGAGC | 70 | 18 |
| | Pleiades | | | |
| | Probe | | | |

The concentration of miR-16 and miR-21 targets were determined, respectively, relative to the 18S rRNA in a HL-60 total RNA sample, shown in FIG. 22a) and FIG. 22b). As shown in these two Figures the biplex assay curves are very similar, indicating efficient biplexing. The Cₜ ratio of miR-16/18S rRNA is about 1.7 and that for miR-21/18S rRNA is bout 1.9. Based on these experiments it appears that the miR-16 and miR-21 concentrations in HL-60 total RNA are similar.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

What is claimed is:

1. A method for amplification of a target nucleic acid in a sample, the method comprising:
(a) contacting a sample suspected of containing the target nucleic acid with an amplification reaction mixture comprising:
at least one flap primer having the formula:
5′-X—Y-3′ (I)
wherein X represents the 5′ sequence portion of the flap primer that is non-complementary to the target nucleic acid, Y represents the 3′ sequence portion of the flap primer that is complementary to the target nucleic acid, wherein X is from 3-40 nucleotides in length;
(b) incubating the reaction mixture under amplification conditions, thereby generating an amplified target nucleic acid; and
(c) optionally detecting the amplified target nucleic acid.

2. The method of claim 1, wherein a signal from the amplified target nucleic acid is at least about 1.25 to about 3-fold greater in comparison to a signal from an amplified target nucleic acid amplified in an amplification reaction mixture that does not comprise at least one flap primer.

3. The method of claim 1, wherein the amount of the amplified target nucleic acid is at least about 1.25 to about 3-fold greater in comparison an amount of an amplified target nucleic acid amplified in an amplification reaction mixture that does not comprise at least one flap primer.

4. The method of claim 1, wherein the reaction mixture comprises a forward flap primer and a reverse flap primer.

5. The method of claim 1, wherein Y comprises a larger number of nucleotides than X.

6. The method of claim 1, wherein X and Y are about an equal number of nucleotides in length.

7. The method of claim 1, wherein Y is more than 10 nucleotides in length.

8. The method of claim 1, wherein X is 9-15 nucleotides in length.

9. The method of claim 8, wherein X is 10-14 nucleotides in length.

10. The method of claim 9, wherein X is 11-13 nucleotides in length.

11. The method of claim 10, wherein X is 12 nucleotides in length.

12. The method of claim 1, wherein X comprises at least 70% adenine or thymine nucleotide bases, or modified bases thereof.

13. The method of claim 12, wherein X comprises at least 80% adenine or thymine nucleotide bases, or modified bases thereof.

14. The method of claim 13, wherein X comprises at least 90% adenine or thymine nucleotide bases, or modified bases thereof.

15. The method of claim 1, wherein the amplification of the target nucleic acid sequence is continuously monitored.

16. The method of claim 1, wherein the amplified target nucleic acid is detected via fluorescence-generating probe.

17. The method of claim 16, wherein the amplified target nucleic acid is detected via a hybridization-based fluorescent probe.

18. The method of claim 16, wherein the amplified target nucleic acid is detected via a DNA binding fluorescent compound.

19. The method of claim 1, wherein the target nucleic acid is selected from the group consisting of DNA, mRNA, tRNA and rRNA.

20. The method of claim 1, wherein the target nucleic acid is less than 30 nucleotides in length.

21. The method of claim 20, wherein the target nucleic acid is miRNA or siRNA.

22. The method of claim 1, further comprising
(c) contacting the amplified target nucleic acid of step (b) with reaction mixture comprising:
(i) a primer comprising a sequence complementary to the target nucleic acid of step (b); and
(ii) a primer comprising a sequence complementary to the target nucleic acid of step (b) and a minor groove binder; and
(d) incubating the reaction mixture of step (c) under amplification conditions, thereby generating a second amplified target nucleic acid; and
(e) optionally detecting the amplified target nucleic acid of step (d).

23. The method of claim 22, wherein the primer of step (c)(i) is a second flap primer of formula I.

24. The method of claim 23, wherein the first primer of formula I and the second flap primer of formula I are the same.

25. The method of claim 22, wherein the primer of step (c)(i) is a MGB-primer.

26. The method of claim 22, wherein the primer of step (c)(ii) is a detection primer further comprising a fluorophore, wherein the fluorophore is quenched by the MGB and insertion of the MGB into a minor groove unquenches the fluorophore.

27. A method for amplification of a target nucleic acid in a sample, the method comprising:
(a) contacting the sample suspected of containing the target nucleic acid with an amplification reaction mixture comprising:
at least one flap primer comprising an annealed helper oligonucleotide and having the formula:
5′-X—Y-3′
3′-X′-5′ (II)
wherein X represents the 5′ sequence portion of the flap primer that is non complementary to the target nucleic acid, X′ represents the helper oligonucleotide sequence that is complementary to X and comprises at least one modified nucleoside base, and Y represents the 3′ sequence portion of the flap primer that is complementary to the target nucleic acid, wherein X is from 3-40 nucleotides in length;
(b) incubating the reaction mixture under amplification conditions, thereby generating an amplified target nucleic acid; and
(c) optionally detecting the amplified target nucleic acid.

28. The method of claim 27, wherein X′ comprises a smaller number of nucleoside bases than X.

29. The method of claim 27, wherein X′ comprises a nucleoside base selected from the group consisting of: 4-(4,6-Diamino-1H-pyrazolo[3,4-d]pyrimidin-3-yl)-but-3-yn-1-ol (Super A); 6-Amino-3-(4-hydroxy-but-1-ynyl)-1,5-dihydro-pyrazolo[3,4-d-]pyrimidin-4-one; 5-(4-hydroxy-but-1-ynyl)-1H-pyrimidine-2,4-dione (Super T), and combinations thereof.

30. The method of claim 27, wherein the reaction mixture comprises a forward flap primer and a reverse flap primer.

31. The method of claim 27, wherein the forward flap primer and the reverse flap primer are independently selected from the group consisting of: Formula I and Formula II.

32. The method of claim 27, wherein Y comprises a larger number of nucleotides than X.

33. The method of claim 27, wherein X and Y are about an equal number of nucleotides in length.

34. The method of claim 27, wherein Y is more than 10 nucleotides in length.

35. The method of claim 27, wherein X is 9-15 nucleotides in length.

36. The method of claim 35, wherein X is 10-14 nucleotides in length.

37. The method of claim 36, wherein X is 11-13 nucleotides in length.

38. The method of claim 37, wherein X is 12 nucleotides in length.

39. The method of claim 27, wherein X comprises at least 70% adenine or thymine nucleotide bases, or modified bases thereof.

40. The method of claim 39, wherein X comprises at least 80% adenine or thymine nucleotide bases, or modified bases thereof.

41. The method of claim 40, wherein X comprises at least 90% adenine or thymine nucleotide bases, or modified bases thereof.

42. The method of claim 27, wherein the amplification of the target nucleic acid sequence is continuously monitored.

43. The method of claim 27, wherein the amplified target nucleic acid is detected via fluorescence-generating probe.

44. The method of claim 43, wherein the amplified target nucleic acid is detected via a hybridization-based fluorescent probe.

45. The method of claim 43, wherein the amplified target nucleic acid is detected via a DNA binding fluorescent compound.

46. The method of claim 27, wherein the target nucleic acid is selected from the group consisting of DNA, mRNA, tRNA and rRNA.

47. The method of claim 27, wherein the target nucleic acid is less than 30 nucleotides in length.

48. The method of claim 47, wherein the target nucleic acid is selected from the group consisting of siRNA and miRNA.

49. The method of claim 27, further comprising
(c) contacting the amplified target nucleic acid of step (b) with reaction mixture comprising:
(i) a primer comprising a sequence complementary to the target nucleic acid of step (b); and
(ii) a primer comprising a sequence complementary to the target nucleic acid of step (b)and a minor groove binder; and
(d) incubating the reaction mixture of step (c) under amplification conditions, thereby generating a second amplified target nucleic acid; and
(e) optionally detecting the amplified target nucleic acid of step (d).

50. The method of claim 49, wherein the primer of step (c)(i) is a second flap primer of formula I.

51. The method of claim 50, wherein the first flap primer of formula I and the second flap primer of formula I are the same.

52. The method of claim 49, wherein the primer of step (c)(i) is a MGB-primer.

53. The method of claim 49, wherein the primer of step (c)(ii) is a detection primer further comprising a fluorophore, wherein the fluorophore is quenched by the MGB and insertion of the MGB into a minor groove unquenches the fluorophore.

54. A method for amplification of a target nucleic acid in a sample, the method comprising:
(a) contacting a sample suspected of containing the target nucleic acid with an amplification reaction mixture comprising:
a detection primer comprising a sequence complementary to the target nucleic acid, a minor groove binder, and fluorophore, wherein the fluorophore is quenched by the MGB and insertion of the MGB into a minor groove unquenches the fluorophore;
(b) incubating the reaction mixture under amplification conditions, thereby generating an amplified target nucleic acid; and
(c) optionally detecting the amplified target nucleic acid.

55. The method of claim 54, wherein the target nucleic acid is selected from the group consisting of DNA, mRNA, tRNA and rRNA.

56. The method of claim 54, wherein the target nucleic acid is less than 30 nucleotides in length.

57. The method of claim 56, wherein the target nucleic acid is selected from the group consisting of siRNA and miRNA.


SEQUENCE LISTING (101 sequences)

SEQ ID NO: 1 - DNA, 15 bp, Artificial Sequence
  Eclipse Probe #1, 5′-minor groove binder-quencher oligonucleotide-fluorophore-3′ (5′-MGB-Q-ODN-FL) probe
  modified_base (1)...(1): n = a modified by dihydrocyclopyrroloindole tripeptide (DPI-3) (MGB) and Eclipse Dark Quencher (Q)
  modified_base (6)...(6): n = ppG, PPG, or Super G, guanine analog 6-amino-1H-pyrazolo[3,4-d]pyrimidin-4(5H)-one
  modified_base (12)...(12): n = Super A, 4,(4,6-diamino-1H-pyrazolo[3,4-d]pyrimidin-3-yl)-but-3-yn-1-ol
  modified_base (15)...(15): n = a modified by 6-carboxyfluorescein (FAM)
ngtagntgtggntgn

SEQ ID NO: 2 - DNA, 16 bp, Artificial Sequence
  Pleiades Probe #2, 5′-minor groove binder-fluorophore-oligonucleotide-quencher-3′ (5′-MGB-FL-ODN-Q) probe
  modified_base (1)...(1): n = ppG, PPG, or Super G, guanine analog 6-amino-1H-pyrazolo[3,4-d]pyrimidin-4(5H)-one modified by dihydrocyclopyrroloindole tripeptide (DPI-3) (MGB) and 6-carboxyfluorescein (FAM)
  modified_base (4)...(4): n = ppG, PPG, or Super G, guanine analog 6-amino-1H-pyrazolo[3,4-d]pyrimidin-4(5H)-one
  modified_base (7)...(7): n = ppG, PPG, or Super G, guanine analog 6-amino-1H-pyrazolo[3,4-d]pyrimidin-4(5H)-one
  modified_base (8)...(8): n = Super A, 4,(4,6-diamino-1H-pyrazolo[3,4-d]pyrimidin-3-yl)-but-3-yn-1-ol
  modified_base (10)...(10): n = ppG, PPG, or Super G, guanine analog 6-amino-1H-pyrazolo[3,4-d]pyrimidin-4(5H)-one
  modified_base (12)...(13): n = ppG, PPG, or Super G, guanine analog 6-amino-1H-pyrazolo[3,4-d]pyrimidin-4(5H)-one
  modified_base (16)...(16): n = ppG, PPG, or Super G, guanine analog 6-amino-1H-pyrazolo[3,4-d]pyrimidin-4(5H)-one modified by Eclipse Dark Quencher (Q)
naantanntntnnatn

SEQ ID NO: 3 - DNA, 33 bp, Artificial Sequence
  Sense Primer #3-F-12
aataaatcataagcctttctttcattccctctc

SEQ ID NO: 4 - DNA, 30 bp, Artificial Sequence
  Sense Primer #3-F-9
aaatcataagcctttctttcattccctctc

SEQ ID NO: 5 - DNA, 24 bp, Artificial Sequence
  Sense Primer #5
atatttgatccacacacgttggtc

SEQ ID NO: 6 - DNA, 27 bp, Artificial Sequence
  Sense Primer #3-F-6
tcataagcctttctttcattccctctc

SEQ ID NO: 7 - DNA, 24 bp, Artificial Sequence
  Sense Primer #3-F-3
taagcctttctttcattccctctc

SEQ ID NO: 8 - DNA, 21 bp, Artificial Sequence
  Sense Primer #3-F-0
gcctttctttcattccctctc

SEQ ID NO: 9 - DNA, 34 bp, Artificial Sequence
  Anti-sense Primer #4-F-12
aataaatcataactgaagcaacatcctgacagtc

SEQ ID NO: 10 - DNA, 31 bp, Artificial Sequence
  Anti-sense Primer #4-F-9
aaatcataactgaagcaacatcctgacagtc

SEQ ID NO: 11 - DNA, 28 bp, Artificial Sequence
  Anti-sense Primer #4-F-6
tcataactgaagcaacatcctgacagtc

SEQ ID NO: 12 - DNA, 25 bp, Artificial Sequence
  Anti-sense Primer #4-F-3
taactgaagcaacatcctgacagtc

SEQ ID NO: 13 - DNA, 22 bp, Artificial Sequence
  Anti-sense Primer #4-F-0
ctgaagcaacatcctgacagtc

SEQ ID NO: 14 - DNA, 36 bp, Artificial Sequence
  Sense Flap Primer #5-F-12
aataaatcataaatatttgatccacacacgttggtc

SEQ ID NO: 15 - DNA, 22 bp, Artificial Sequence
  Anti-sense Primer #6
aaggtatttgagcggcttcctc

SEQ ID NO: 16 - DNA, 34 bp, Artificial Sequence
  Anit-sense Flap Primer #6-F-12
aataaatcataaaaggtatttgagcggcttcctc

SEQ ID NO: 17 - DNA, 17 bp, Artificial Sequence
  Eclipse Probe #7, 5′-minor groove binder-quencher oligonucleotide-fluorophore-3′ (5′-MGB-Q-ODN-FL) probe
  modified_base (1)...(1): n = g modified by dihydrocyclopyrroloindole tripeptide (DPI-3) (MGB) and Eclipse Dark Quencher (Q)
  modified_base (8)...(8): n = Super A, 4,(4,6-diamino-1H-pyrazolo[3,4-d]pyrimidin-3-yl)-but-3-yn-1-ol
  modified_base (17)...(17): n = g modified by 6-carboxyfluorescein (FAM)
ncagaaancaaaacagn

SEQ ID NO: 18 - DNA, 18 bp, Artificial Sequence
  Pleiades Probe #8, 5′-minor groove binder-fluorophore-oligonucleotide-quencher-3′ (5′-MGB-FL-ODN-Q) probe
  modified_base (1)...(1): n = ppG, PPG, or Super G, guanine analog 6-amino-1H-pyrazolo[3,4-d]pyrimidin-4(5H)-one modified by dihydrocyclopyrroloindole tripeptide (DPI-3) (MGB) and 6-carboxyfluorescein (FAM)
  modified_base (10)...(10): n = ppG, PPG, or Super G, guanine analog 6-amino-1H-pyrazolo[3,4-d]pyrimidin-4(5H)-one
  modified_base (12)...(12): n = ppG, PPG, or Super G, guanine analog 6-amino-1H-pyrazolo[3,4-d]pyrimidin-4(5H)-one
  modified_base (15)...(15): n = ppG, PPG, or Super G, guanine analog 6-amino-1H-pyrazolo[3,4-d]pyrimidin-4(5H)-one
  modified_base (17)...(17): n = ppG, PPG, or Super G, guanine analog 6-amino-1H-pyrazolo[3,4-d]pyrimidin-4(5H)-one
  modified_base (18)...(18): n = c modified by Eclipse Dark Quencher (Q)
nttttaaccntnctnann

SEQ ID NO: 19 - DNA, 129 bp, Artificial Sequence
  amplicon target in MGB Eclipse and Pleiades assays
aataaatcataaatatttgatccacacacgttggtcttttaaccgtgctgagcagaaaacaaaacaggttaagaagagccgggtggcagctgacagaggaagccgctcaaataccttttatgatttatt

SEQ ID NO: 20 - DNA, 21 bp, Artificial Sequence
  Sense Primer #9
gcctttctttcattccctctc

SEQ ID NO: 21 - DNA, 33 bp, Artificial Sequence; identical to SEQ ID NO: 3
  Sense Flap Primer #9-F-12

SEQ ID NO: 22 - DNA, 22 bp, Artificial Sequence
  Sense Primer #10
ctgaagcaacatcctgacagtc

SEQ ID NO: 23 - DNA, 34 bp, Artificial Sequence; identical to SEQ ID NO: 9
  Sense Primer #10-F-12

SEQ ID NO: 24 - DNA, 15 bp, Artificial Sequence
  Eclipse Probe #11, 5′-minor groove binder-quencher oligonucleotide-fluorophore-3′ (5′-MGB-Q-ODN-FL) probe
  modified_base (1)...(1): n = a modified by dihydrocyclopyrroloindole tripeptide (DPI-3) (MGB) and Eclipse Dark Quencher (Q)
  modified_base (6)...(6): n = Super A, 4,(4,6-diamino-1H-pyrazolo[3,4-d]pyrimidin-3-yl)-but-3-yn-1-ol
  modified_base (12)...(12): n = Super A, 4,(4,6-diamino-1H-pyrazolo[3,4-d]pyrimidin-3-yl)-but-3-yn-1-ol
  modified_base (15)...(15): n = a modified by 6-carboxyfluorescein (FAM)
ngtagntgtggntgn

SEQ ID NO: 25 - DNA, 16 bp, Artificial Sequence
  Pleiades Probe #12, 5′-minor groove binder-fluorophore-oligonucleotide-quencher-3′ (5′-MGB-FL-ODN-Q) probe
  modified_base (1)...(1): n = ppG, PPG, or Super G, guanine analog 6-amino-1H-pyrazolo[3,4-d]pyrimidin-4(5H)-one modified by dihydrocyclopyrroloindole tripeptide (DPI-3) (MGB) and 6-carboxyfluorescein (FAM)
  modified_base (4)...(4): n = ppG, PPG, or Super G, guanine analog 6-amino-1H-pyrazolo[3,4-d]pyrimidin-4(5H)-one
  modified_base (7)...(7): n = ppG, PPG, or Super G, guanine analog 6-amino-1H-pyrazolo[3,4-d]pyrimidin-4(5H)-one
  modified_base (8)...(8): n = Super A, 4,(4,6-diamino-1H-pyrazolo[3,4-d]pyrimidin-3-yl)-but-3-yn-1-ol
  modified_base (10)...(10): n = ppG, PPG, or Super G, guanine analog 6-amino-1H-pyrazolo[3,4-d]pyrimidin-4(5H)-one
  modified_base (12)...(13): n = ppG, PPG, or Super G, guanine analog 6-amino-1H-pyrazolo[3,4-d]pyrimidin-4(5H)-one
  modified_base (16)...(16): n = ppG, PPG, or Super G, guanine analog 6-amino-1H-pyrazolo[3,4-d]pyrimidin-4(5H)-one modified by Eclipse Dark Quencher (Q)
naantanntntnnatn

SEQ ID NO: 26 - DNA, 110 bp, Artificial Sequence
  Amplified Target
aataaatcataagcctttctttcattccctctctgaaaagtattccaacgtgatattccttgaagtagatgtggatgactgtcaggatgttgcttcagttatgatttatt

SEQ ID NO: 27 - DNA, 26 bp, Artificial Sequence
  Primer #13
taatgaaggtagtctaacacatcctg

SEQ ID NO: 28 - DNA, 18 bp, Artificial Sequence
  SPrimer #14-F-0
gtagtctaacacatcctg

SEQ ID NO: 29 - DNA, 30 bp, Artificial Sequence
  FPrimer #14-F-12
aataaatcataagtagtctaacacatcctg

SEQ ID NO: 30 - DNA, 18 bp, Artificial Sequence
  SPrimer #15-F-0
  modified_base (8)...(8): n = Super A, 4,(4,6-diamino-1H-pyrazolo[3,4-d]pyrimidin-3-yl)-but-3-yn-1-ol
gtagtctnacacatcctg

SEQ ID NO: 31 - DNA, 30 bp, Artificial Sequence
  SPrimer #15-F-12
  modified_base (20)...(20): n = Super A, 4,(4,6-diamino-1H-pyrazolo[3,4-d]pyrimidin-3-yl)-but-3-yn-1-ol
aataaatcataagtagtctnacacatcctg

SEQ ID NO: 32 - DNA, 18 bp, Artificial Sequence
  SPrimer #16-F-0
  modified_base (8)...(8): n = Super A, 4,(4,6-diamino-1H-pyrazolo[3,4-d]pyrimidin-3-yl)-but-3-yn-1-ol
  modified_base (13)...(13): n = Super A, 4,(4,6-diamino-1H-pyrazolo[3,4-d]pyrimidin-3-yl)-but-3-yn-1-ol
gtagtctnacacntcctg

SEQ ID NO: 33 - DNA, 30 bp, Artificial Sequence
  FPrimer #16-F-12
  modified_base (20)...(20): n = Super A, 4,(4,6-diamino-1H-pyrazolo[3,4-d]pyrimidin-3-yl)-but-3-yn-1-ol
  modified_base (25)...(25): n = Super A, 4,(4,6-diamino-1H-pyrazolo[3,4-d]pyrimidin-3-yl)-but-3-yn-1-ol
aataaatcataagtagtctnacacntcctg

SEQ ID NO: 34 - DNA, 32 bp, Artificial Sequence
  Primer #17
ccccaatatctcattattacctggaccaagtc

SEQ ID NO: 35 - DNA, 18 bp, Artificial Sequence
  SPrimer #18-F-0
attacctggaccaagtct

SEQ ID NO: 36 - DNA, 30 bp, Artificial Sequence
  FPrimer #18-F-12
aataaatcataaattacctggaccaagtct

SEQ ID NO: 37 - DNA, 18 bp, Artificial Sequence
  SPrimer #19-F-0
  modified_base (10)...(10): n = Super A, 4,(4,6-diamino-1H-pyrazolo[3,4-d]pyrimidin-3-yl)-but-3-yn-1-ol
attacctggnccaagtct

SEQ ID NO: 38 - DNA, 30 bp, Artificial Sequence
  FPrimer #19-F12
  modified_base (22)...(22): n = Super A, 4,(4,6-diamino-1H-pyrazolo[3,4-d]pyrimidin-3-yl)-but-3-yn-1-ol
aataaatcataaattacctggnccaagtct

SEQ ID NO: 39 - DNA, 17 bp, Artificial Sequence
  Probe #20, 5′-minor groove binder-quencher oligonucleotide-fluorophore-3′ (5′-MGB-Q-ODN-FL) probe
  modified_base (1)...(1): n = ppG, PPG, or Super G, guanine analog 6-amino-1H-pyrazolo[3,4-d]pyrimidin-4(5H)-one modified by dihydropyrroloindole tripeptide (DPI-3) (MGB) and Eclipse Dark Quencher (Q)
  modified_base (11)...(11): n = Super A, 4,(4,6-diamino-1H-pyrazolo[3,4-d]pyrimidin-3-yl)-but-3-yn-1-ol
  modified_base (16)...(16): n = Super A, 4,(4,6-diamino-1H-pyrazolo[3,4-d]pyrimidin-3-yl)-but-3-yn-1-ol
  modified_base (17)...(17): n = g modified by 6-carboxyfluorescein (FAM)
nttgatccagnaagtnn

SEQ ID NO: 40 - DNA, 12 bp, Artificial Sequence
  Flap sequence #21
aataaatcataa

SEQ ID NO: 41 - DNA, 82 bp, Artificial Sequence
  human parainfluenza RNA Amplicon
taatgaaggtagtctaacacatcctgagattgtggttgatccagaaagtagacttggtccaggtaataatgagatattgggg

SEQ ID NO: 42 - DNA, 29 bp, Artificial Sequence
  beta-2-microglobulin (B2MG) housekeeping gene Primer #22
  modified_base (13)...(13): n = Super A, 4,(4,6-diamino-1H-pyrazolo[3,4-d]pyrimidin-3-yl)-but-3-yn-1-ol
  modified_base (17)...(17): n = Super A, 4,(4,6-diamino-1H-pyrazolo[3,4-d]pyrimidin-3-yl)-but-3-yn-1-ol
  modified_base (22)...(22): n = Super A, 4,(4,6-diamino-1H-pyrazolo[3,4-d]pyrimidin-3-yl)-but-3-yn-1-ol
gcctgccgtgtgnaccntgtgnctttgtc

SEQ ID NO: 43 - DNA, 22 bp, Artificial Sequence
  beta-2-microglobulin (B2MG) housekeeping gene Primer #23
cggcatcttcaaacctccatga

SEQ ID NO: 44 - DNA, 41 bp, Artificial Sequence
  beta-2-microglobulin (B2MG) housekeeping gene Flap Primer #22-F-12
  modified_base (25)...(25): n = Super A, 4,(4,6-diamino-1H-pyrazolo[3,4-d]pyrimidin-3-yl)-but-3-yn-1-ol
  modified_base (29)...(29): n = Super A, 4,(4,6-diamino-1H-pyrazolo[3,4-d]pyrimidin-3-yl)-but-3-yn-1-ol
  modified_base (34)...(34): n = Super A, 4,(4,6-diamino-1H-pyrazolo[3,4-d]pyrimidin-3-yl)-but-3-yn-1-ol
aataaatcataagcctgccgtgtgnaccntgtgnctttgtc

SEQ ID NO: 45 - DNA, 34 bp, Artificial Sequence
  beta-2-microglobulin (B2MG) housekeeping gene Flap Primer #23-F-12
aataaatcataacggcatcttcaaacctccatga

SEQ ID NO: 46 - DNA, 17 bp, Artificial Sequence
  beta-2-microglobulin (B2MG) housekeeping gene Probe #24, 5′-minor groove binder-quencher oligonucleotide-fluorophore-3′ (5′-MGB-Q-ODN-FL) probe
  modified_base (1)...(1): n = g modified by dihydrocyclopyrroloindole tripeptide (DPI-3) (MGB) and Eclipse Dark Quencher (Q)
  modified_base (17)...(17): n = a modified by 6-carboxyfluorescein (FAM)
nttaagtgggatcgagn

SEQ ID NO: 47 - DNA, 22 bp, Artificial Sequence
  gene of interest human mitogen-activated protein kinase 3 (MAP2K3) Primer #25
caattccagaggacatccttgg

SEQ ID NO: 48 - DNA, 25 bp, Artificial Sequence
  gene of interest human mitogen-activated protein kinase 3 (MAP2K3) Primer #26
taaggacattggagggcttcacatc

SEQ ID NO: 49 - DNA, 34 bp, Artificial Sequence
  gene of interest human mitogen-activated protein kinase 3 (MAP2K3) Flap Primer #25-F-12
aataaatcataacaattccagaggacatccttgg

SEQ ID NO: 50 - DNA, 37 bp, Artificial Sequence
  gene of interest human mitogen-activated protein kinase 3 (MAP2K3) Flap Primer #26-F-12
aataaatcataataaggacattggagggcttcacatc

SEQ ID NO: 51 - DNA, 17 bp, Artificial Sequence
  gene of interest human mitogen-activated protein kinase 3 (MAP2K3) Probe #27, 5′-minor groove binder-quencher oligonucleotide-fluorophore-3′ (5′-MGB-Q-ODN-FL) probe
  modified_base (1)...(1): n = c modified by dihydrocyclopyrroloindole tripeptide (DPI-3) (MGB) and Eclipse Dark Quencher (Q)
  modified_base (17)...(17): n = g modified by fluorophore TET
ntgtgtctatcgtgcgn

SEQ ID NO: 52 - DNA, 10 bp, Artificial Sequence
  hsa-miR-139 Primer #22
tctacagtgc

SEQ ID NO: 53 - DNA, 22 bp, Artificial Sequence
  hsa-miR-139 Flap Primer #22-F-12
aataaatcataatctacagtgc

SEQ ID NO: 54 - DNA, 10 bp, Artificial Sequence
  hsa-miR-139 Primer #23
  modified_base (1)...(1): n = Super T, 5-(4-hydroxy-but-1-ynyl)-1H-pyrimidine-2,4-dione
  modified_base (3)...(3): n = Super T, 5-(4-hydroxy-but-1-ynyl)-1H-pyrimidine-2,4-dione
  modified_base (4)...(4): n = Super A, 4,(4,6-diamino-1H-pyrazolo[3,4-d]pyrimidin-3-yl)-but-3-yn-1-ol
  modified_base (6)...(6): n = Super A, 4,(4,6-diamino-1H-pyrazolo[3,4-d]pyrimidin-3-yl)-but-3-yn-1-ol
  modified_base (8)...(8): n = Super T, 5-(4-hydroxy-but-1-ynyl)-1H-pyrimidine-2,4-dione
ncnncngngc

SEQ ID NO: 55 - DNA, 10 bp, Artificial Sequence
  hsa-miR-139 Primer #24
agacacgtgc

SEQ ID NO: 56 - DNA, 22 bp, Artificial Sequence
  hsa-miR-139 Flap Primer #24-F-12
aataaatcataaagacacgtgc

SEQ ID NO: 57 - DNA, 10 bp, Artificial Sequence
  hsa-miR-139 Primer #25
  modified_base (1)...(1): n = Super A, 4,(4,6-diamino-1H-pyrazolo[3,4-d]pyrimidin-3-yl)-but-3-yn-1-ol
  modified_base (3)...(3): n = Super A, 4,(4,6-diamino-1H-pyrazolo[3,4-d]pyrimidin-3-yl)-but-3-yn-1-ol
  modified_base (5)...(5): n = Super A, 4,(4,6-diamino-1H-pyrazolo[3,4-d]pyrimidin-3-yl)-but-3-yn-1-ol
ngncncgtgc

SEQ ID NO: 58 - DNA, 18 bp, Artificial Sequence
  hsa-miR-139 DNA target, miRNA hsa-miR-139 target, Target sequence
tctacagtgcacgtgtct

SEQ ID NO: 59 - RNA, 23 nt, Artificial Sequence
  hsa-miR-142-3P RNA Target
uguaguguuuccuacuuuaugga

SEQ ID NO: 60 - DNA, 16 bp, Artificial Sequence
  hsa-miR-142-3P Limiting Primer #1
  modified_base (1)...(1): n = t modified by dihydrocyclopyrroloindole dipeptide (DPI-2)
ngtagtgtttcctact

SEQ ID NO: 61 - DNA, 16 bp, Artificial Sequence
  hsa-miR-142-3P Limiting Primer #2
  modified_base (1)...(1): n = t modified by dihydrocyclopyrroloindole tripeptide (DPI-3)
ngtagtgtttcctact

SEQ ID NO: 62 - DNA, 28 bp, Artificial Sequence
  hsa-miR-142-3P Excess Primer #3
aataaatcataagtggacggtccgaggt

SEQ ID NO: 63 - DNA, 38 bp, Artificial Sequence
  hsa-miR-142-3P RT Primer #4
gtggacggtccgaggtctggatacgactccataaagta

SEQ ID NO: 64 - DNA, 36 bp, Artificial Sequence
  hsa-miR-142-3P RT Primer #4a
gtggacggtccgaggtctggatacgactccataaag

SEQ ID NO: 65 - DNA, 17 bp, Artificial Sequence
  hsa-miR-142-3P Pleiades probe #5
  modified_base (8)...(8): n = Super A, 4,(4,6-diamino-1H-pyrazolo[3,4-d]pyrimidin-3-yl)-but-3-yn-1-ol
tttatggngtcgtatcc

SEQ ID NO: 66 - DNA, 12 bp, Artificial Sequence
  RT Helper Oligo #6
  modified_base (2)...(2): n = Super T, 5-(4-hydroxy-but-1-ynyl)-1H-pyrimidine-2,4-dione
  modified_base (6)...(6): n = Super A, 4,(4,6-diamino-1H-pyrazolo[3,4-d]pyrimidin-3-yl)-but-3-yn-1-ol
  modified_base (10)...(10): n = Super A, 4,(4,6-diamino-1H-pyrazolo[3,4-d]pyrimidin-3-yl)-but-3-yn-1-ol
gncgtntccnga

SEQ ID NO: 67 - DNA, 28 bp, Artificial Sequence
  hsa-miR-142-3P Limiting Primer #7
gcccgctcataatgtagtgtttcctact

SEQ ID NO: 68 - DNA, 19 bp, Artificial Sequence
  hsa-miR-142-3P Limiting Primer #8
gacggtccgaggtctggat

SEQ ID NO: 69 - RNA, 65 nt, Artificial Sequence
  hsa-miR-16-1 precursor DNA target
uagcagcacguaaauauuggcguuaagauucuaaaauuaucuccaguauuaacugugcugcugaa

SEQ ID NO: 70 - DNA, 25 bp, Artificial Sequence
  hsa-miR-16-1 precursor Sense Primer #9
  modified_base (14)...(14): n = Super T, 5-(4-hydroxy-but-1-ynyl)-1H-pyrimidine-2,4-dione
  modified_base (20)...(20): n = Super A, 4,(4,6-diamino-1H-pyrazolo[3,4-d]pyrimidin-3-yl)-but-3-yn-1-ol
cccgctcataatcnccagtnttaac

SEQ ID NO: 71 - DNA, 23 bp, Artificial Sequence
  hsa-miR-16-1 precursor Antisense Primer #10
cgcattgatcaatcgtacggtct

SEQ ID NO: 72 - DNA, 40 bp, Artificial Sequence
  hsa-miR-16-1 precursor RT Primer #11
cgcattgatcaatcgtacggtctctggatacgactcagca

SEQ ID NO: 73 - DNA, 16 bp, Artificial Sequence
  hsa-miR-16-1 precursor Pleiades Probe #2
  modified_base (5)...(5): n = Super T, 5-(4-hydroxy-but-1-ynyl)-1H-pyrimidine-2,4-dione
  modified_base (9)...(9): n = Super A, 4,(4,6-diamino-1H-pyrazolo[3,4-d]pyrimidin-3-yl)-but-3-yn-1-ol
  modified_base (13)...(13): n = Super A, 4,(4,6-diamino-1H-pyrazolo[3,4-d]pyrimidin-3-yl)-but-3-yn-1-ol
tgagncgtntccngag

SEQ ID NO: 74 - RNA, 22 nt, Artificial Sequence
  hsa-miR-16 mature sequence target
uagcagcacguaaauauuggcg

SEQ ID NO: 75 - DNA, 23 bp, Artificial Sequence
  hsa-miR-16 mature sequence Sense Primer #13
cccgctcataatagcagcacgta

SEQ ID NO: 76 - DNA, 28 bp, Artificial Sequence
  hsa-miR-16 mature sequence Antisense Primer #14
aataaatcataagtggacggtccgaggt

SEQ ID NO: 77 - DNA, 33 bp, Artificial Sequence
  hsa-miR-16 mature sequence RT Primer #15
gtggacggtccgaggtctggatacgaccgccaa

SEQ ID NO: 78 - DNA, 38 bp, Artificial Sequence
  hsa-miR-16 mature sequence RT Primer #15a
gtggacggtccgaggtctggatacgaccgccaatattt

SEQ ID NO: 79 - DNA, 17 bp, Artificial Sequence
  hsa-miR-16 mature sequence Pleiades Probe #16
  modified_base (1)...(1): n = ppG, PPG, or Super G, guanine analog 6-amino-1H-pyrazolo[3,4-d]pyrimidin-4(5H)-one
  modified_base (5)...(5): n = Super A, 4,(4,6-diamino-1H-pyrazolo[3,4-d]pyrimidin-3-yl)-but-3-yn-1-ol
  modified_base (7)...(7): n = Super A, 4,(4,6-diamino-1H-pyrazolo[3,4-d]pyrimidin-3-yl)-but-3-yn-1-ol
  modified_base (11)...(11): n = Super A, 4,(4,6-diamino-1H-pyrazolo[3,4-d]pyrimidin-3-yl)-but-3-yn-1-ol
nccantntttncgtgct

SEQ ID NO: 80 - DNA, 24 bp, Artificial Sequence
  synthetic miRNA template let-7a Sense Primer #17
cccgctcataatgaggtagtaggt

SEQ ID NO: 81 - DNA, 28 bp, Artificial Sequence
  synthetic miRNA template let-7a Antisense Primer #18
aataaatcataagtggacggtccgaggt

SEQ ID NO: 82 - DNA, 38 bp, Artificial Sequence
  synthetic miRNA template let-7a RT Primer #19
gtggacggtccgaggtctggatacgacaactatacaac

SEQ ID NO: 83 - DNA, 18 bp, Artificial Sequence
  synthetic miRNA template let-7a Pleiades Probe #20
  modified_base (11)...(11): n = Super A, 4,(4,6-diamino-1H-pyrazolo[3,4-d]pyrimidin-3-yl)-but-3-yn-1-ol
  modified_base (15)...(15): n = Super A, 4,(4,6-diamino-1H-pyrazolo[3,4-d]pyrimidin-3-yl)-but-3-yn-1-ol
  modified_base (17)...(17): n = Super A, 4,(4,6-diamino-1H-pyrazolo[3,4-d]pyrimidin-3-yl)-but-3-yn-1-ol
tggatacgacnactntnc

SEQ ID NO: 84 - DNA, 33 bp, Artificial Sequence
  synthetic miRNA template let-7d RT Primer
gtggacggtccgaggtctggatacgacaactat

SEQ ID NO: 85 - RNA, 22 nt, Artificial Sequence
  synthetic miRNA template target let-7a
ugagguaguagguuguauaguu

SEQ ID NO: 86 - RNA, 21 nt, Artificial Sequence
  synthetic miRNA template target let-7d
agagguaguagguugcauagu

SEQ ID NO: 87 - DNA, 20 bp, Artificial Sequence
  hsa-miR-16 mature sequence Sense Primer #21
  modified_base (15)...(15): n = Super A, 4,(4,6-diamino-1H-pyrazolo[3,4-d]pyrimidin-3-yl)-but-3-yn-1-ol
aataaatcataagcngcacg

SEQ ID NO: 88 - DNA, 21 bp, Artificial Sequence
  hsa-miR-16 mature sequence Antisense Labeled Primer #22
  modified_base (1)...(1): n = a modified by dihydrocyclopyrroloindole dipeptide (DPI-2) and 6-carboxyfluorescein (FAM)
  modified_base (17)...(17): n = Super A, 4,(4,6-diamino-1H-pyrazolo[3,4-d]pyrimidin-3-yl)-but-3-yn-1-ol
  modified_base (19)...(19): n = Super T, 5-(4-hydroxy-but-1-ynyl)-1H-pyrimidine-2,4-dione
nataaatcataacgccnanat

SEQ ID NO: 89 - DNA, 24 bp, Artificial Sequence
  hsa-miR-16 mature sequence RT Primer #23
  modified_base (17)...(17): n = Super A, 4,(4,6-diamino-1H-pyrazolo[3,4-d]pyrimidin-3-yl)-but-3-yn-1-ol
  modified_base (22)...(23): n = Super T, 5-(4-hydroxy-but-1-ynyl)-1H-pyrimidine-2,4-dione
aataaatcataacgccnatatnna

SEQ ID NO: 90 - DNA, 23 bp, Artificial Sequence
  hsa-miR-16 mature sequence miR-16 Sense Primer #24
cccgctcataatagcagcacgta

SEQ ID NO: 91 - DNA, 28 bp, Artificial Sequence
  hsa-miR-16 mature sequence miR-16 Antisense Primer #25
aataaatcataagtggacggtccgaggt

SEQ ID NO: 92 - DNA, 33 bp, Artificial Sequence; identical to SEQ ID NO: 77
  hsa-miR-16 mature sequence miR-16 RT Primer #26

SEQ ID NO: 93 - DNA, 16 bp, Artificial Sequence
  hsa-miR-16 mature sequence miR-16 Pleiades Probe #27
aaatattggcggtcgt

SEQ ID NO: 94 - DNA, 32 bp, Artificial Sequence
  18S rRNA 18S Sense Primer #29
  modified_base (14)...(14): n = Super A, 4,(4,6-diamino-1H-pyrazolo[3,4-d]pyrimidin-3-yl)-but-3-yn-1-ol
  modified_base (20)...(20): n = Super A, 4,(4,6-diamino-1H-pyrazolo[3,4-d]pyrimidin-3-yl)-but-3-yn-1-ol
  modified_base (25)...(25): n = Super A, 4,(4,6-diamino-1H-pyrazolo[3,4-d]pyrimidin-3-yl)-but-3-yn-1-ol
aataaatcataagncacggncaggnttgacag

SEQ ID NO: 95 - DNA, 30 bp, Artificial Sequence
  18S rRNA 18S Antisense Primer/RT Primer #30
  modified_base (18)...(18): n = Super A, 4,(4,6-diamino-1H-pyrazolo[3,4-d]pyrimidin-3-yl)-but-3-yn-1-ol
  modified_base (23)...(23): n = Super A, 4,(4,6-diamino-1H-pyrazolo[3,4-d]pyrimidin-3-yl)-but-3-yn-1-ol
aataaatcataaaattanccagncaaatcg

SEQ ID NO: 96 - DNA, 18 bp, Artificial Sequence
  18S rRNA 18S Pleiades Probe #31
  modified_base (1)...(1): n = Super A, 4,(4,6-diamino-1H-pyrazolo[3,4-d]pyrimidin-3-yl)-but-3-yn-1-ol
  modified_base (7)...(7): n = Super A, 4,(4,6-diamino-1H-pyrazolo[3,4-d]pyrimidin-3-yl)-but-3-yn-1-ol
nttcttngttggtggagc

SEQ ID NO: 97 - RNA, 22 nt, Artificial Sequence
  hsa-miR-21 target sequence
uagcuuaucagacugauguuga

SEQ ID NO: 98 - DNA, 24 bp, Artificial Sequence
  hsa-miR-21 miR-21 Sense Primer #32
gcccgctagcttatcagactgatg

SEQ ID NO: 99 - DNA, 28 bp, Artificial Sequence
  hsa-miR-21 miR-21 Antisense Primer #33
aataaatcataagtggacggtccgaggt

SEQ ID NO: 100 - DNA, 17 bp, Artificial Sequence
  hsa-miR-21 miR-21 Pleiades Probe #34
  modified_base (1)...(1): n = ppG, PPG, or Super G, guanine analog 6-amino-1H-pyrazolo[3,4-d]pyrimidin-4(5H)-one
  modified_base (8)...(8): n = Super A, 4,(4,6-diamino-1H-pyrazolo[3,4-d]pyrimidin-3-yl)-but-3-yn-1-ol
  modified_base (15)...(15): n = Super A, 4,(4,6-diamino-1H-pyrazolo[3,4-d]pyrimidin-3-yl)-but-3-yn-1-ol
ngatacgnctcaacntc

SEQ ID NO: 101 - DNA, 33 bp, Artificial Sequence
  hsa-miR-21 miR-21 RT Primer #35
gtggacggtccgaggtctggatacgactcaaca